SEQUENCE LISTING (37 sequences)

SEQ ID NO: 1 - DNA, 21 bp, Homo sapiens
aaaaaaaaaaatttttttttt

SEQ ID NO: 2 - DNA, 74 bp, Homo sapiens
cacacacacacacacacacactctctctctcacacacacacactcagtcacacacacacacacacacacacaca

SEQ ID NO: 3 - DNA, 33 bp, Homo sapiens
acacacacacacacatacttctttttttttttt

SEQ ID NO: 4 - DNA, 45 bp, Homo sapiens
tatatatatatatatatatatatattttttttttttttttttttt

SEQ ID NO: 5 - DNA, 35 bp, Homo sapiens
aaaaaaaaaaaaaaaaaaacaaacaaaaaaaaaaa

SEQ ID NO: 6 - DNA, 32 bp, Homo sapiens
tttttttttttttttttgtgtgtgtgtgtgtg

SEQ ID NO: 7 - DNA, 28 bp, Homo sapiens
aaaaaaaaaaatatatatatatatatat

SEQ ID NO: 8 - DNA, 77 bp, Homo sapiens
atatatatatattcttttctctatacatttatgcaaacttgcatttgatgacatcatattttgcaggtttttttttt

SEQ ID NO: 9 - DNA, 29 bp, Homo sapiens
ttttttttttctttttctttttttttttt

SEQ ID NO: 10 - DNA, 56 bp, Homo sapiens
tgtgtgtgtgtgtgtgtgagagagagagagagagagacagagacagagagagagag

SEQ ID NO: 11 - DNA, 68 bp, Homo sapiens
ttttttttttacaagaccatttttcattatgaatttgtaccatgtgtcagcacctttttttttttttt

SEQ ID NO: 12 - DNA, 60 bp, Homo sapiens
gatggatggatggatggatggatggatggatggatggatggacggacggacggacggacg

SEQ ID NO: 13 - DNA, 38 bp, Homo sapiens
caccaccaccaccaccatgcccaccaccaccaccacca

SEQ ID NO: 14 - DNA, 45 bp, Homo sapiens
cagcagcagcagcagcagcagcaacagcagcagcagcagcagcag

SEQ ID NO: 15 - DNA, 25 bp, Homo sapiens
aaaaaaaaaaaacaaaaaaaaaaaa

SEQ ID NO: 16 - DNA, 42 bp, Homo sapiens
acacacacacacacacacacacacacaccacacacacacaca

SEQ ID NO: 17 - DNA, 22 bp, Homo sapiens
aaaaaaaaaaagaaaaaaaaaa

SEQ ID NO: 18 - DNA, 43 bp, Homo sapiens
ctctctctctctctctatatgtgtgtgtgtgtatatatatata

SEQ ID NO: 19 - DNA, 40 bp, Homo sapiens
tgtgtgtgtgtgtgtgtgagagagagagagagagagagag

SEQ ID NO: 20 - DNA, 79 bp, Homo sapiens
tgtgtgtgtgtgtgtatgtatgtgtatatatatatatatctatatatatatagatatagatagatagatagatagatag

SEQ ID NO: 21 - DNA, 31 bp, Homo sapiens
aaaaaaaaaaaaagaaaaagaaaaaaaaaaa

SEQ ID NO: 22 - DNA, 32 bp, Homo sapiens
tatatatatatatatatatatatttttttttt

SEQ ID NO: 23 - DNA, 75 bp, Homo sapiens
ttttttttttcaatccattcagacaacttttgttgttgttgttgttgttttgtgtttttcggtgttttttttttt

SEQ ID NO: 24 - DNA, 57 bp, Homo sapiens
gctgctgctgctgctgctgctgaagttgctgttgctgttgcagctgctgctgctgct

SEQ ID NO: 25 - DNA, 57 bp, Homo sapiens
atgatgatgatgatgatgatgatgataatgatgatagctatgatgatgatgatgatg

SEQ ID NO: 26 - DNA, 65 bp, Homo sapiens
aaaaaaaaaaaattatatatatatatatatatatatttcgtggcaattttttttttttttttttt

SEQ ID NO: 27 - DNA, 36 bp, Homo sapiens
tttttttttttcaaactttctctttttttttttttt

SEQ ID NO: 28 - DNA, 41 bp, Homo sapiens
aaaaaaaaaaaaaagggaatagatactaaaaaaaaaaaaaa

SEQ ID NO: 29 - DNA, 27 bp, Homo sapiens
ttttttttttttccttttttttttttt

SEQ ID NO: 30 - DNA, 39 bp, Homo sapiens
tttttttttttttttttttttttttttgagagagagaga

SEQ ID NO: 31 - DNA, 43 bp, Homo sapiens
tgtgtgtgtgtgtgtgtgttttttttttttttttttttttttt

SEQ ID NO: 32 - DNA, 22 bp, Homo sapiens
tttttttttttaaaaaaaaaaa

SEQ ID NO: 33 - DNA, 47 bp, Homo sapiens
aaaaaaaaaaaagaaaaaaaaaagaaaagaagaagaagaagaagaag

SEQ ID NO: 34 - DNA, 56 bp, Homo sapiens
acacacacacacgcgcgcgcgcgcacacacacacacacacacacacacacacacac

SEQ ID NO: 35 - DNA, 56 bp, Homo sapiens
tctgtctgtctgtctgtctgtctctctctctctctctctctatatatatatatata

SEQ ID NO: 36 - DNA, 67 bp, Homo sapiens
gagagagagagagagagagagggaaataaataaataaataaataaataaataaataaataaataaat

SEQ ID NO: 37 - DNA, 50 bp, Homo sapiens
tgtgtgtgtgtgtgtgtgtgtgttttttcccccccccccttttttttttt

(19) United States
(12) Patent Application Publication
(10) Pub. No.: US 2023/0230661 A1
(43) Pub. Date: Jul. 20, 2023

(54) MICROSATELLITE INSTABILITY DETERMINING METHOD AND SYSTEM THEREOF

(71) Applicants: Shu-jen CHEN, Taipei (TW); ACT Genomics (IP) Limited, Hong Kong (HK)

(72) Inventors: YA-CHI YEH, Taipei (TW); CHIEN-HUNG CHEN, Taipei (TW); SHU-JEN CHEN, Taipei (TW); YING-JA CHEN, Taipei (TW); KUAN-YING CHEN, Taipei (TW)

(21) Appl. No.: 18/002,054

(22) PCT Filed: Jun. 18, 2021
(86) PCT No.: PCT/US2021/037969

§ 371 Date: Dec. 17, 2022

Related U.S. Application Data
(60) Provisional application No. 63/041,103, filed on Jun. 18, 2020.

(51) Int. Cl. G16B 40/20; G16B 20/00

(52) U.S. Cl. CPC G16B 40/20; G16B 20/00

(57) ABSTRACT

A method and a system used to determine microsatellite instability (MSI) status utilizing Next-Generation Sequencing (NGS) and a machine learning model are disclosed. The present disclosure further provides a method and a system for identifying a treatment based on the computed MSI status data for the human subject.

### CROSS-REFERENCE TO RELATED APPLICATIONS

This application claims priority of Provisional Application No. 63/041,103, filed on Jun. 18, 2020, the content of which is incorporated herein in its entirety by reference.

### REFERENCE TO A “SEQUENCE LISTING”, A TABLE, OR A COMPUTER PROGRAM LISTING APPENDIX SUBMITTTED ON A COMPACT DISC AND AN INCORPORATION-BY-REFERENCE OF THE MATERIAL ON THE COMPACT DISC

The instant application contains a Sequence Listing which has been submitted electronically in ASCII format and is hereby incorporated by reference in its entirety. Said ASCII copy, created Jun. 11, 2021, is named “ACTG-7PCT_ST25.txt” and is 6,293 bytes in size

### BACKGROUND OF THE INVENTION

This disclosure is related to the fields of molecular diagnostics, cancer genomics, and molecular biology.

Microsatellite instability (MSI) is a molecular phenotype indicative of underlying genomic hypermutability. The gain or loss of nucleotides from microsatellite tracts can arise from impairments in the mismatch repair (MMR) system, limiting the correction of spontaneous mutations in repetitive DNA sequences. MSI-affected tumors may, accordingly, be caused by mutational inactivation or epigenetic silencing of genes in the MMR pathway. MSI has been associated with improved prognosis. The ability of MSI to predict pembrolizumab response has led to the first tumor-agnostic drug approval by the FDA in May 2017. Additional evidence showed an improved response for microsatellite instability-high (MSI-H) patients to the anti-PD-1 agents nivolumab and MED10680, the anti-PD-L1 agent durvalumab, and the anti-CTLA-4 agent ipilimumab. With these results, MSI-H has been approved as the molecular marker for immune checkpoint inhibitors.

MSI is typically detected through PCR assay (MSI-PCR) by fragment analysis (FA) using the peak pattern of five microsatellite loci to determine the MSI status of individual samples. Samples with two or more unstable microsatellites are referred to as MSI-High, whereas samples with one or no unstable microsatellite detected are referred to as MSS. However, since each microsatellite locus should be evaluated by comparing the paired tumor and normal tissue, MSI-PCR assay is not always feasible for cases with limited tissue samples, especially the sample containing few normal cells. Immunohistochemistry (IHC) is another typical assay that may be used for MSI status detection. It detects samples with MSI through MMR protein expression testing. However, MMR-IHC cannot always detect loss of mutated proteins resulting from missense mutations and may have normal staining even for some protein-truncating mutations. Further, interpretation of both MSI-PCR and IHC data is manual and qualitative. There is a need in the art for developing a quantitative assay to determine the MSI status efficiently and accurately for patients. Currently several next-generation sequencing (NGS) assays are found to be feasible to determine MSI status. In general, NGS-based MSI testing offers the advantage of providing automated analysis based on quantitative statistics, which reduces analysis time and the variation derived from inter-observer and inter-laboratory compared to MSI-PCR assay. However, some NGS-based MSI-detection methods such as MANTIS and MSIsensor require a matched-normal sample for the evaluation. For other methods, e.g., MSIplus, though do not require a matched-normal sample in the assay, further improvement like adding more microsatellite loci may be needed. There is still space for improving NGS-based MSI testing

### SUMMARY OF THE INVENTION

The present disclosure provides improved techniques for determining MSI status. The present disclosure uses a trained machine learning model to determine MSI status from large-panel clinical targeted NGS data accounting for at least six microsatellite loci, and preferably at least one hundred microsatellite loci. The trained machine learning model uses different weights on the different features, e.g., peak width, peak height, peak location, and simple sequence repeat (SSR) type, to achieve high robustness and efficiency for MSI status detection from NGS data without matched normal sample. Furthermore, through validating the trained machine learning model using an independent dataset of clinical samples across various cancer types, the trained machine learning model is proved to have high sensitivity and specificity for MSI status detection.

In one general aspect, the disclosure relates to a method of generating a model for predicting a MSI status, including:
(a) collecting a clinical sample and an estimated MSI status data thereof;
(b) sequencing, through NGS, at least six microsatellite loci of the clinical sample to generate sequencing data;
(c) extracting a MSI feature from the sequencing data;
(d) training a machine learning model by mapping a MSI feature data with the estimated MSI status data; and
(e) outputting a trained machine learning model.

In some embodiments, the MSI feature data is calculated by a baseline. In some embodiments, the baseline for calculating the MSI feature data is established by normal samples or samples with MSS status. In some embodiments, the baseline is established from the mean of each the MSI feature of each SSR region across the normal samples. Preferably, the baseline is established from the mean peak width of each SSR region.

In some embodiments, the estimated MSI status data is retrieved from a cancer patient through known assay method including but not limited to MSI-PCR assay, IHC, NGS-based MSI testing including MANTIS, MSIsensor, MSIplus, or Large Panel NGS. In some embodiments, the MSI status is microsatellite stability (MSS) or MSI-H. In some embodiments, the MSI features include peak width, peak height, peak location, SSR type, or any combination thereof.

In some embodiments, the machine learning model includes but is not limited to regression-based models, tree-based models, Bayesian models, support vector machines, boosting models, or neural network-based models. In some embodiments, the machine learning model includes but is not limited to a logistic regression model, a random forest model, an extremely randomized trees model, a polynomial regression model, a linear regression model, a gradient descent model, and an extreme gradient boost model.

In some embodiments, the trained machine learning model includes a defined weight of each microsatellite locus. In some embodiments, the trained machine learning model includes a defined weight of the MSI feature in each microsatellite locus. The trained machine learning model is predictive of MSI status.

In some embodiments, the machine learning model has a cutoff value of 0.1, 0.15, 0.2, 0.25, 0.3, 0.35, 0.4, 0.45, or 0.5.

In some embodiments, the estimated MSI status data or the computed MSI status data indicates microsatellite stability (MSS) or microsatellite instability-high (MSI-H).

In another general aspect, the disclosure relates to a computer-implemented method for determining MSI status, including:
(a) collecting a clinical sample from a subject;
(b) sequencing, through NGS, at least six microsatellite loci of the clinical sample so as to generate a sequencing data;
(c) extracting a MSI feature from the sequencing data;
(d) inputting a MSI feature data into the trained machine learning model; and
(e) generating a computed MSI status.

In some embodiments, the computer-implemented method further includes step (f): outputting the computed MSI status data to an electronic storage medium or a display.

In some embodiments, the method further includes a step of identifying a treatment for a subject based on the computed MSI status data and/or administering a therapeutically effective amount of treatment to the subject.

In some embodiments, the treatment includes but is not limited to surgery, individual therapy, chemotherapy, radiation therapy, immunotherapy, or any combination thereof. In some embodiments, the immunotherapy includes administering the drug including but not limited to anti-PD-1 agents pembrolizumab, nivolumab and MED10680, anti-PD-L1 agent durvalumab, and anti-CTLA-4 agent ipilimumab.

In some embodiments, the microsatellite loci is at least 7, 10, 15, 20, 30, 40, 50, 100, 150, 200, 250, 300, 350, 400, 450, 500, 550, or 600 loci. In some embodiments, the microsatellite loci are identifying by sequencing SSR regions in the chromosomal regions. In some embodiments, the microsatellite loci are excluded due to low coverage, unstable peak call, high variability in peak width, or low weight. In some embodiments, the microsatellite loci with high variability in peak width has a peak width greater than 2 in 5 replicate runs, 3 in 6 replicate runs, 3 in 7 replicate runs, 3 in 8 replicate runs, 3 in 9 replicate runs, or 4 in 10 replicate runs.

In some embodiments, the sample originates from a cell line, biopsy, primary tissue, frozen tissue, formalin-fixed paraffin-embedded (FFPE), liquid biopsy, blood, serum, plasma, buffy coat, body fluid, visceral fluid, ascites, paracentesis, cerebrospinal fluid, saliva, urine, tears, seminal fluid, vaginal fluid, aspirate, lavage, buccal swab, circulating tumor cell (CTC), cell-free DNA (cfDNA), circulating tumor DNA (ctDNA), DNA, RNA, nucleic acid, purified nucleic acid, purified DNA, or purified RNA.

In some embodiments, the sample is a clinical sample. In some embodiments, the sample originates from a diseased patient. In some embodiments, the sample originates from a patient having cancer, solid tumor, hematologic malignancy, rare genetic disease, complex disease, diabetes, cardiovascular disease, liver disease, or neurological disease. In some embodiments, the sample originates from a patient having Adenocarcinoma, Adenoid cystic carcinoma, Adrenal cortical carcinoma, Ampulla Vater cancer, Anal cancer, Appendix cancer, Basal ganglia glioma, Bladder cancer, Brain cancer, Brain tumor glioma, Breast cancer, Buccal cancer, Cervical cancer, Cholangiocarcinoma, Chondrosarcoma, Clear cell carcinoma, Colon cancer, Colorectal cancer, Cystic duct carcinoma, Dedifferentiated liposarcoma, Desmoid, Diffuse midline glioma, Endometrial cancer, Endometrioid adenocarcinoma, Epithelioid rhabdomyosarcoma, Esophageal cancer, Extraskeletal chondroblastic osteosarcoma, Eyelid sebaceous carcinoma, Fallopian tube cancer, Gallbladder cancer, Gastric Cancer, Gastrointestinal stromal tumor, Glioblastoma multiforme, Head and Neck Cancers, Hepatocellular carcinoma, High grade glioma, Hypopharyngeal Cancer, Intima sarcoma, Infantile fibrosarcoma, Invasive ductal carcinoma, Kidney cancer, Leiomyosarcoma, Liposarcoma, Liver angiosarcoma, Liver cancer, Lung cancer, Melanoma, Metastasis of unknown origin, Nasopharyngeal cancer, NSCLC adenocarcinoma, Oesophageal cancer, Oral Cancer, Oropharyngeal cancer, Osteosarcoma, Ovarian cancer, Pancreatic cancer, Papillary Thyroid Carcinoma, Peritoneal cancer, Primary peritoneal serous carcinoma, Prostate cancer, Rectal cancer, Renal cancer, Salivary gland cancer, Sarcomatoid Carcinoma, Sigmoid cancer, Sinus cancer, Skin cancer, Soft tissue sarcoma, Squamous cell carcinoma, Stomach adenoacrinoma, Submandibular gland cancer, Thymic cancer, Thymoma involvement, Thyroid cancer, Tongue cancer, Tonsillar cancer, Transitional cell carcinoma, Uterine cancer, Uterine sarcoma, or Uterus leiomyosarcoma. In some embodiments, the sample originates from a pregnant woman, a child, an adolescent, an elder, or an adult. In some embodiments, the sample is a research sample. In some embodiments, the sample originates from a group of samples. In some embodiments, the group of samples is from related species. In some embodiments, the group of samples is from different species.

In some embodiments, the machine learning model is trained by using a training set having MSI status data and MSI feature data.

In some embodiments, the NGS system includes but not limited to the MiSeq, HiSeq, MiniSeq, iSeq, NextSeq, and NovaSeq sequencers manufactured by Illumina, Inc., Ion Personal Genome Machine (PGM), Ion Proton, Ion S5 series, and Ion GeneStudio S5 series manufactured by Life Technologies, Inc., BGlseq series, DNBseq series and MGlseq series, manufactured by BGI, and MinION/PromethION sequencers manufactured by Oxford Nanopore Technologies.

In some embodiments, the sequencing reads are generated from nucleic acids that are amplified from the original sample or the nucleic acids captured by the bait. In some embodiments, the sequencing reads are generated from a sequencer that required the addition of an adapter sequence. In some embodiments, the sequencing reads are generated from a method that includes but is not limited to hybrid capture, primer extension target enrichment, a molecular inversion probe-based method, or multiplex target-specific PCR.

In another general aspect, the disclosure relates to a system for determining MSI status. The system includes a data storage device storing instructions for determining characteristics of MSI status and a processor configured to execute the instructions to perform a method. Further, the method includes the following steps:
(a) training a machine learning model, wherein the machine learning model maps the training data of one or more MSI features with the training estimated MSI status;
(b) collecting a clinical sample from a human subject;
(c) sequencing at least six microsatellite loci of the clinical sample to generate a sequence data by using NGS;
(d) computing the estimated MSI status by inputting a MSI features data extracting from the sequencing data into the trained machine learning model; and
(e) outputting the computed MSI status data.

### BRIEF DESCRIPTION OF DRAWINGS

One or more embodiments are illustrated by ways of example, and not by limitation, in the figures of the accompanying drawings, wherein elements are having the same reference numeral designations represent like elements throughout. The drawings are not to scale unless otherwise disclosed.

FIGS. 1(a)-(c) are schematic diagrams illustrating the parameters used to characterize microsatellite instability.

FIG. 2 is a ROC curve of the MSI model.

FIG. 3 is Box plot of the MSI score in the validation data set.

The drawings are only schematic and are non-limiting. In the drawings, the size of some of the elements may be exaggerated and not drawn on scale for illustrative purposes. The dimensions and the relative dimensions do not necessarily correspond to actual reductions to the practice of the disclosure.

### DETAILED DESCRIPTION OF THE INVENTION

The making and using of the embodiments of the disclosure are discussed in detail below. It should be appreciated, however, that the embodiments provide many applicable inventive concepts that can be embodied in a wide variety of specific contexts. The specific embodiments discussed are merely illustrative of specific ways to make and use the embodiments and do not limit the scope of the disclosure.

Unless defined otherwise, all technical and scientific terms used herein have the same meaning as commonly understood by a person skilled in the art to which this disclosure belongs. As used herein, the singular forms “a,” “an,” and “the” include plural referents unless the context clearly dictates otherwise.

As used herein, “microsatellite” means a tract of repetitive DNA in which certain DNA motifs are repeated. “Microsatellite loci” refers to the regions of the microsatellite. The terms “microsatellite” and “SSR,” as well as “microsatellite loci” and “SSR region” are used interchangeably, respectively, where the context allows. In some embodiments of the disclosure, type of microsatellite loci or SSR region refers to mono-, di-, tri-, tetra, or pentanucleotide repeats or certain complex nucleotide type in a nucleotide sequence. Preferably, type of the microsatellite loci or SSR region refers to mononucleotide with at least ten repeats, dinucleotide with at least six repeats, trinucleotide with at least five repeats, tetranucleotide with at least five repeats, pentanucleotide with at least five repeats, and the complex nucleotide type including but not limited to SEQ ID NOs: 1-37.

As used herein, “MSI status” or “MMR status” refers to the presence of “MSI” or “unstable microsatellite (loci),” a clonal or somatic change in the number of repeated DNA nucleotide units in microsatellites. The present disclosure estimates the MSI status as MSS or MSI-H. “MSI-H” refers to those in which the number of repeats present in microsatellite loci differs significantly from the number of repeats that are in the DNA of a normal cell. “MSS” refers to those who have no functional defects in DNA MMR and have no significant differences between tumor and normal cell in microsatellite loci.

As used herein, “cutoff value” or “threshold” refers to a numerical value or other representation whose value is used to arbitrate between two or more states of classification for a biological sample. In some embodiments of the disclosure, the cutoff value is set according to the training result of the machine learning model and is used to distinguish between MSI-H and MSS. If the MSI score is greater than the cutoff value, the MSI status is determined as MSI-H; or if the MSI score is less than the cutoff value, the MSI status is determined as MSS.

As used herein, “peak” refers to a microsatellite distribution pattern in the microsatellite loci. The peak may be analyzed using data generated by next-generation sequencing, where the number of allele repeat length within each microsatellite locus is considered as peak width, the read counts of the most frequently observed allele is referred to as peak height, and the location difference between the peak height in each microsatellite locus of tumor tissue and reference genome is referred to as peak location. In some embodiments of the disclosure, peak width, peak height, or peak location are used as MSI features to estimate the MSI status.

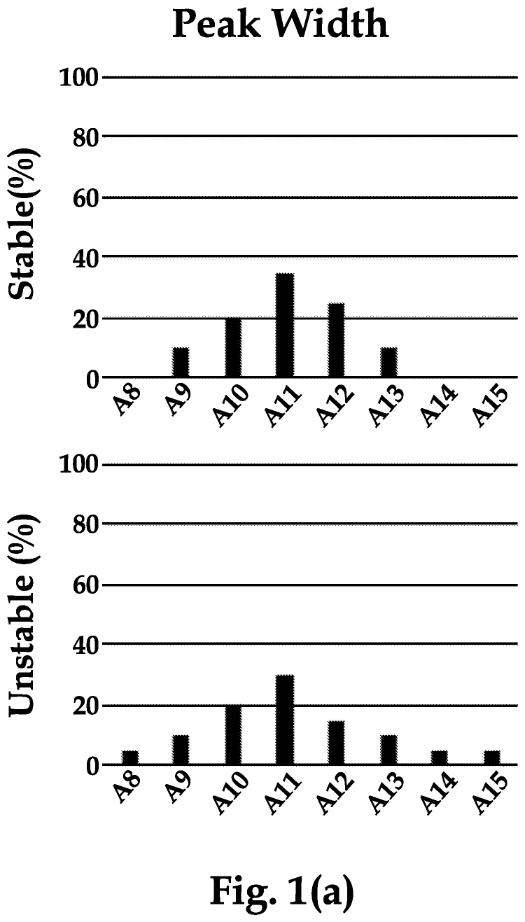

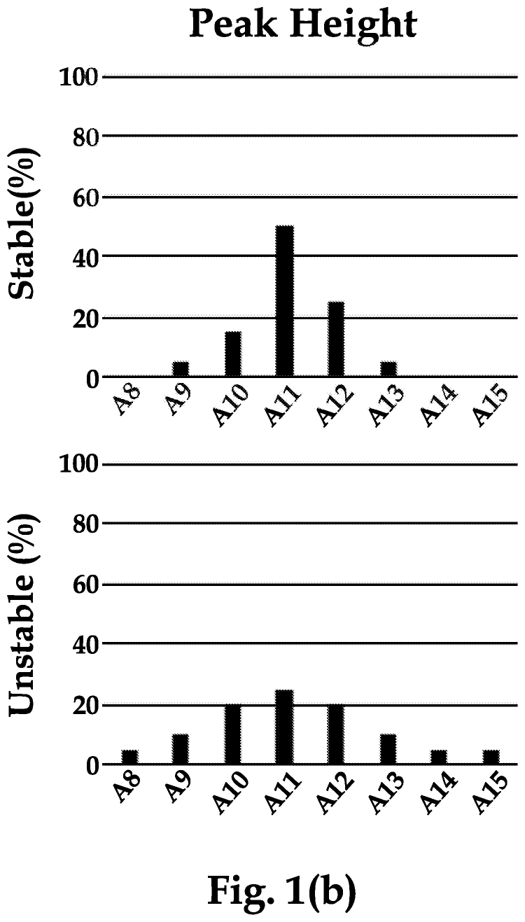

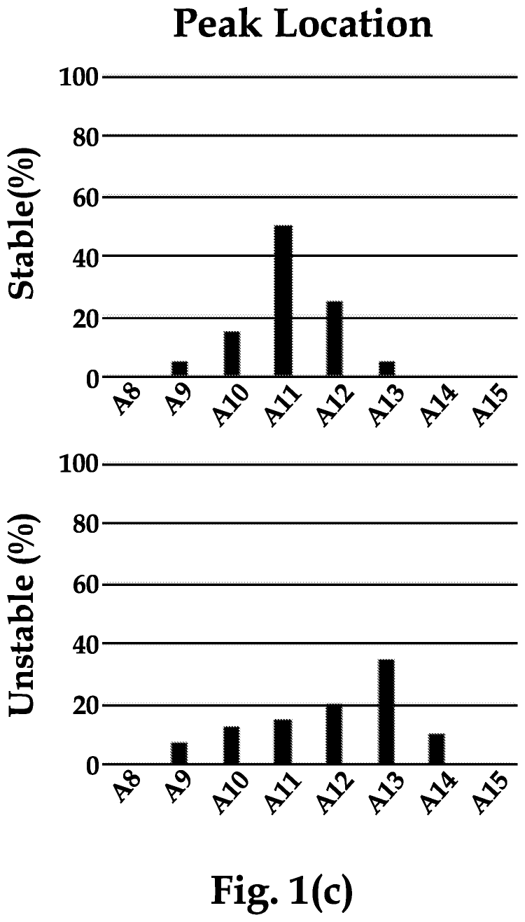

As shown in FIGS. 1(a) to 1(c), each locus is a short sequence repeat. When detected by PCR followed by Sanger sequencing or by Next-Generation Sequencing (NGS) methods, each microsatellite locus shows a pattern of a peak. A peak can be characterized by its peak width, peak height, and peak location. When a microsatellite locus becomes unstable, the peak width, peak height, and/or peak location may change. Here, the x-axis shows the alleles for each peak signal. For example, in FIG. 1(a), the first signal shows an allele with eight repeats of nucleotide A at that microsatellite locus. This peak has a peak width of 5, peak height of about 35%, and peak location at 11 A. Peak location can also be described by its chromosome position, such as chr4:55598211. The y-axis shows the percentage of reading count for a given peak signal as compared to the other peak signals. Therefore, the sum of peak height for a given peak is one. FIG. 1(a) shows the peak distribution when the peak width is widened from 5 to 8 when this locus becomes unstable. FIG. 1(b) shows that when a peak is unstable, the peak height may become lower. In this example, it went from 50% to 25%. FIG. 1(c) shows that when a peak is unstable, the peak location may change. In this example, it changed from 10 As to 12 As.

Generally, to understand the MSI status, a matched paired analysis would be performed to identify microsatellite loci in the tumor that are different compared to matched normal tissue. “Matched normal tissue” or “normal pair tissue” as used herein refers to normal tissue from the same patient. However, in some embodiments of the disclosure, the machine learning model detects MSI status from NGS data without matched normal tissue. A pooled normal sample is used to establish the mean of each the MSI feature of each SSR region across the normal population as a baseline for MSI detection. Data from individual clinical tumor tissue will be compared to the peak pattern of the baseline data to determine microsatellite status for each SSR region in that sample.

As used herein, “tumor purity” is the proportion of cancer cells in a tumor sample. Tumor purity impacts the accurate assessment of molecular and genomics features as assayed with NGS approaches. In some embodiments of the disclosure, the clinical sample has a tumor purity at least 5, 10, 15, 20, 25, 30, 35, 40, 45, 50, 55, 60, 65, 70, 75, 80, 85, 90, 95, or 100%. Preferably, the present disclosure disclosure identifies the sample within the tumor purity at least 20%.

As used herein, “depth” or “total depth” refers to the number of sequencing reads per location. “Mean depth,” “mean total depth,” or “total mean depth” refers to the average number of reads across the entire sequencing region. Generally, the total mean depth has an impact on the performance of the NGS assay. The higher the mean total depth, the lower the variability in the variant frequency of the variant. In some embodiments of the disclosure, the mean depth of the sample across the entire sequencing region is at least 200x, 300x, 400, 500x, 600x, 700x, 800x, 900x, 1000x, 2000x, 3000x, 4000x, 5000x, 6000x, 8000x, 10000x, or 20000x. Preferably, the mean depth of the sample across the entire sequencing region is at least 500x.

As used herein, “coverage” refers to the total depth at a given locus and can be used interchangeably with “depth.” In some embodiments of the disclosure, “low coverage” means the read depth lower than 5x, 10x, 15x, 20x, 25x, 30x, 35x, 40x, 45x, or 50x from a sample on a locus.

As used herein, “target base coverage” refers to the percentage of the sequenced region that is sequenced at a depth above a predefined value. Target base coverage needs to specify the depth at which it is evaluated. In some embodiments, the target base coverage at 100x is 85%. That means 85% of the target sequenced bases is covered by at least 100x depth of sequencing reads. In some embodiments, the target base coverage at 30x, 40x, 50x, 60x, 70x, 80x, 90x, 100x, 125x, 150x, 175x, 200x, 300x, 400x, 500x, 750x, 1000x is above 70%, 75%, 80%, 85%, 90%, or 95%.

As used herein, “human subject” refers to those with formally diagnosed disorders, those without formally recognized disorders, those receiving medical attention, those at risk of developing the disorders, etc.

As used herein, “treat,” “treatment,” and “treating” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors.

As used herein, “therapeutically effective amount” means an amount of a therapeutically active molecule needed to elicit the desired biological or clinical effect. In preferred embodiments of the disclosure, “a therapeutically effective amount” is the amount of drug needed to treat cancer patients with MSI-H.

The present disclosure is further illustrated by the following Examples, which are provided for the purpose of demonstration rather than limitation.

### EXAMPLE 1

### Training a Machine Learning Model for Detection of MSI Status

Formalin-fixed paraffin-embedded (FFPE) samples were prepared from cancer patients through surgical or needle biopsy samples. Genomic DNA was extracted using QIAamp DNA FFPE Tissue Kit (QIAGEN, Hilden, Germany). Eighty nanograms of DNA were amplified using multiplexed PCR targeting a panel of 440 genes and 1.8 Mbps. The samples were sequenced by using Ion Proton or Ion S5 Prime (Thermo Fisher Scientific, Waltham, Mass.) system with the Ion PI or 540 Chip (Thermo Fisher Scientific, Waltham, Mass.) following manufacturer recommended protocol. Raw sequence reads were processed by the manufacturer-provided software Torrent Variant Caller (TVC) v5.2, and .bam and .vcf files were generated.

(1) Candidate Loci Selection

Using the MIcroSAtellite identification tool (MISA, Beier, Thiel, Munch, Scholz, & Mascher,

2017), SSR regions in the chromosomal regions covered by the ACTOnco Panel assay were identified. A total of 600 SSR regions, including mononucleotide with at least ten repeats, dinucleotide with at least six repeats, trinucleotide with at least five repeats, tetranucleotide with at least five repeats, pentanucleotide with at least five repeats, and complex nucleotide type, were identified by MISA. The sequences of the complex SSR regions are provided in Table 1.

TABLE 1
Complex microsate11ite loci

| SEQ | | |
|---|---|---|
| ID | | Size |
| NO | Microsatellite sequence | (bp) |
| 1 | (A)11(T)10 | 21 |
| 2 | (CA)10ctctctctct(CA)6ctcagt(CA)13 | 74 |
| 3 | (AC)7atacttc(T)12 | 33 |
| 4 | (TA)12(T)21 | 45 |
| 5 | (A)19caaac(A)11 | 35 |
| 6 | (T)16(TG)8 | 32 |
| 7 | (A)10(AT)9 | 28 |
| 8 | (AT)6tcttttctctatacatttatgcaaactt | 77 |
| | g(T)10catttgatgacatcatattttgcagg | |
| 9 | (T)10ctttttc(T)12 | 29 |
| 10 | (TG)9(AG)9acagagac(AG)6 | 56 |
| 11 | (T)10acaagaccatttttcattatgaatttg | 68 |
| | taccatgtgtcagcacc(T)14 | |
| 12 | (GATG)10(GACG)5 | 60 |
| 13 | (CAC)5catgc(CCA)6 | 38 |
| 14 | (CAG)7caa(CAG)7 | 45 |
| 15 | (A)12c(A)12 | 25 |
| 16 | (AC)14(CA)7 | 42 |
| 17 | (A)11g(A)10 | 22 |
| 18 | (CT)8ata(TG)6(TA)6 | 43 |
| 19 | (TG)9(AG)11 | 40 |
| 20 | (TG)7tatgtatgtg(TA)7tc(TA)6gat | 79 |
| | (ATAG)6 | |
| 21 | (A)13gaaaaag(A)11 | 31 |
| 22 | (TA)11(T)10 | 32 |
| 23 | (T)10caatccattcagacaactt(TTG)6ttt | 75 |
| | tgtgtttttcggtg(T)11 | |
| 24 | (GCT)7gaagttgctgttgctgttgca(GCT)5 | 57 |
| 25 | (ATG)8ataatgatgatagct(ATG)6 | 57 |
| 26 | (A)12t(TA)11tttcgtggcaa(T)19 | 65 |
| 27 | (T)11caaactttctc(T)14 | 36 |
| 28 | (A)14gggaatagatact(A)14 | 41 |
| 29 | (T)12cc(T)13 | 27 |
| 30 | (T)27(GA)6 | 39 |
| 31 | (TG)9(T)25 | 43 |
| 32 | (T)11(A)11 | 22 |
| 33 | (A)12g(A)10gaa(AAG)7 | 47 |
| 34 | (AC)6(GC)6(AC)16 | 56 |
| 35 | (TCTG)5(TC)10(TA)8 | 56 |
| 36 | (GA)10ggg(AAAT)11 | 67 |
| 37 | (TG)11tttttt(C)11(T)11 | 50 |

Note: The uppercase sequences in parenthesis are the sequences being repeated by the number of times indicated by the number following it. Lowercase sequences not in parenthesis are sequences between two repetition regions within one identified loci.

We first examined the chromosomal location of each SSR region. A total of 34 SSR loci were found located on the X chromosome and were excluded.

In order to develop a robust MSI prediction algorithm for ACTOnco assay, we plan to include only SSR regions from the remaining 566 candidate loci, which shows reproducible peak patterns in clinical FFPE samples in the prediction model. To identify SSRs with good reproducibility across different sequencing runs, we examined the coverage and peak pattern of the 566 SSR regions in a set of 10 FFPE clinical samples across six replicate runs.

In order to include only highly confident reads on each SSR region for the prediction model, a minimum read depth of 30x from a sample on a locus was required. Additionally, to determine the total number of repeats of different lengths (peak width) on a SSR region, a minimum of 5% of allele frequency for a repeated length was required to be included. For example, for a sample on a locus with segments of mononucleotide repeats, if the allele frequencies are detected as 2% for 15 bases, 10% for 16 bases, 20% for 17 bases, 30% for 18 bases, 20% for 19 bases, 10% for 20 bases, and 8% for 21 bases, the total number of repeats of different lengths (peak width) will be 6 with the length of 15 bases uncounted.

We excluded 138 SSR regions due to their low coverage (<30 reads for the SSR region), unstable peak call (missing peak width data in any sequencing run), high variability in peak width (variation in peak width greater than 3 in 6 replicate runs) or low weight (the MSI feature data around the last 5% contributions to the prediction model). The remaining 428 microsatellite loci were used for the subsequent baseline establishment and model training.

(2) Baseline Establishment

Population baseline for all 428 loci was established. The mean peak width of 77 normal samples sequenced in the Ion Proton sequencer was used to establish a baseline. The mean peak width of 81 normal samples sequenced in the Ion S5 Prime sequencer was used to establish another baseline. The MSI baseline was established from the mean peak width of each SSR region across the normal population. The standard deviation of peak width was also calculated for each candidate locus. For a given locus, it is considered unstable if the difference in peak width between a given clinical sample and the baseline falls outside of two times the standard deviation. The total unstable loci percentage is calculated by dividing the number of unstable loci by the total number of loci used.

(3) MSI Prediction Model and Model Validation

A total of 122 colorectal cancer (FFPE samples) sequenced on Ion Proton and Ion S5 Prime were used in training the machine learning model. Of those samples, 76 are MSS, and 46 are MSI-H samples based on a 5-marker MSI-PCR detection system (Promega MSI Analysis System, version 1.2). For each sample, the loci with read depth less than 30x were not considered in model training and were reported as missing information. Additionally, to determine the peak width on a SSR region, a minimum of 5% of allele frequency for a repeated length (allele) was required to be included in training the model. The difference in the peak width between the MSS baseline and clinical samples were used for calculation in the following logistic regression model:

MSI status (MSS/MSI-H)=β0+β1loci1+β2loci2+β3loci3+ . . . +β428loci428 where β is a weight.

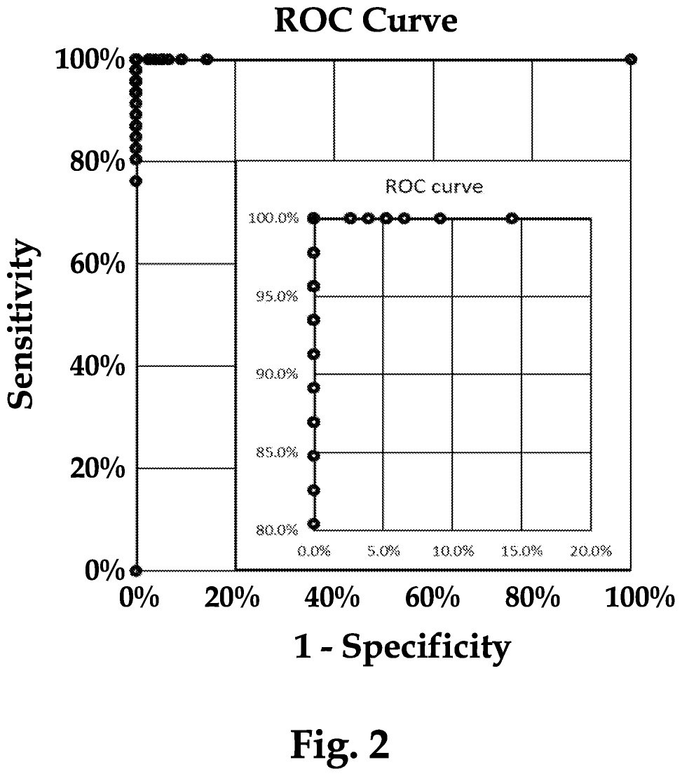

We divided 122 training data by 7:3 ratio for training and testing and randomly assigned samples to train and test the data for 1000 iterations. Due to the small sample size, all 122 training data were used to set the cutoff value. The MSI score used for setting the cutoff value is calculated by selecting the median MSI score for each sample when it is selected as testing data during the 1000 iterations. The ROC curve for the model performance is shown in FIG. 2. According to analysis results, we decided to select 0.15 as the cutoff value of the MSI prediction model to achieve high sensitivity (100%) and specificity (100%).

### EXAMPLE 2

### Using the MSI Model to Determine the MSI Status of Cancer Samples

We next used an independent set of 439 clinical FFPE samples, including 30 MSI-H and 409 MSS samples, to validate the MSI model. Samples include but are not limited to lung cancer, colorectal cancer, breast cancer, ovarian cancer, pancreatic cancer, cholangiocarcinoma, gastric cancer, glioblastoma, sarcoma, cervical cancer, leiomyosarcoma, and liposarcoma. These samples were processed using the same method as described in Example 1 to sequence the 428 loci region to a mean sequencing depth of at least 500x and 85% of the target region reaching a target base coverage of 100x.

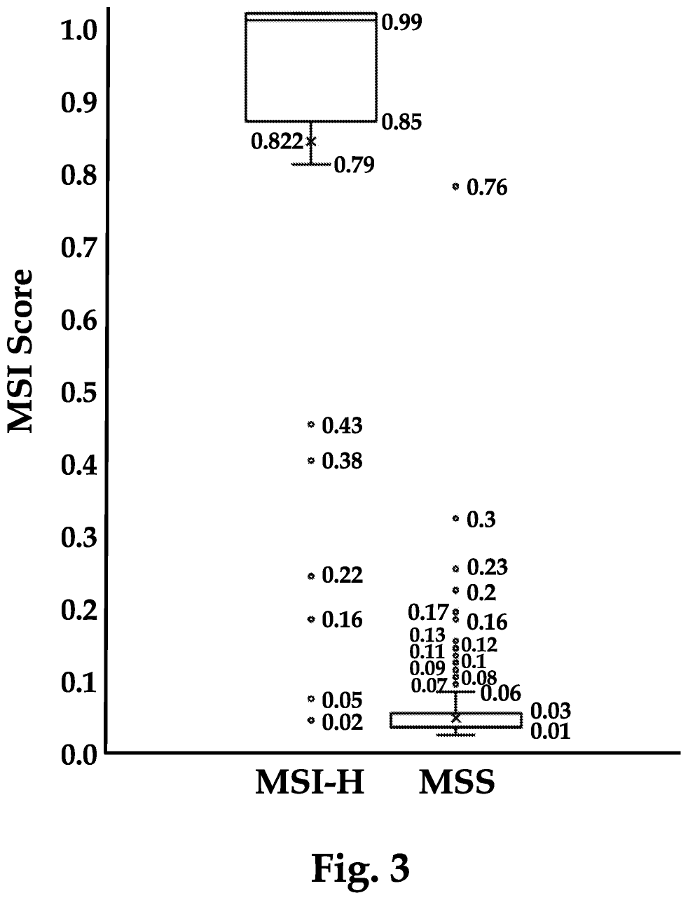

FIG. 3 shows the resulting MSI scores of the MSI-H and MSS samples are clearly distinguished. The results of model validation demonstrate that the positive percent agreement (PPA) and negative percent agreement (NPA) of this model are 93.3% and 98.5%, respectively. The validation results are provided in Tables 2-5.

TABLE 2
MSI detection of clinical samples

| | | | | Target base | | | | |
|---|---|---|---|---|---|---|---|---|
| Sample | | Tumor | Mean | coverage | MSI | MSI Status | Unstable | MSI status |
| ID | Cancer type | purity | depth | at 100x | score | by MSI model | Loci % | by 5-loci PCR |
| F00173 | Lung cancer | NA | 1877 | 0.97 | 0.01 | MSS | 3.49 | MSS |
| F00212 | Oesophagus cancer | 50% | 900.7 | 0.94 | 0.01 | MSS | 3.94 | MSS |
| F01597 | Pancreatic cancer | 60% | 1488 | 0.95 | 0.01 | MSS | 3.59 | MSS |
| F02095 | Adenocarcinoma | NA | 1155 | 0.96 | 0.02 | MSS | 5.01 | MSS |
| F01143 | Lung cancer | 40% | 1127 | 0.96 | 0.06 | MSS | 3.4 | MSS |
| F01407 | Unknown primary | 5% | 1355 | 0.96 | 0 | MSS | 4.81 | MSS |
| E00708 | Adenoid cystic carcinoma | 50% | 1454 | 0.94 | 0.01 | MSS | 4.99 | MSS |
| F01911 | Adenoid cystic carcinoma | 45% | 983.3 | 0.96 | 0.01 | MSS | 3.33 | MSS |
| F02161 | Adenoid cystic carcinoma | 40% | 1238 | 0.97 | 0 | MSS | 3.86 | MSS |
| F01464 | Adrenal cortical carcinoma | 40% | 1174 | 0.96 | 0.01 | MSS | 5.57 | MSS |
| F00249 | Ampulla Vater cancer | 25% | 1097 | 0.96 | 0.01 | MSS | 2.21 | MSS |
| F01517 | Appendix cancer | 90% | 1441 | 0.96 | 0 | MSS | 4.07 | MSI-L |
| F00507 | Brain cancer | 25% | 1142 | 0.96 | 0.03 | MSS | 3.5 | MSS |
| F02040 | Brain cancer | 30% | 2237 | 0.99 | 0.05 | MSS | 5.8 | MSS |
| F01581 | Basal ganglia glioma | 70% | 794.5 | 0.92 | 0.01 | MSS | 3.57 | MSS |
| F01530 | Brain tumor glioma | 40% | 2411 | 0.97 | 0.01 | MSS | 4.58 | MSS |
| F02387 | Breast cancer | NA | 1640 | 0.98 | 0 | MSS | 10.52 | MSI-L |
| F02197 | Breast cancer | 20% | 1226 | 0.95 | 0.02 | MSS | 5.14 | MSS |
| E00086 | Breast cancer | 55% | 1064 | 0.94 | 0.01 | MSS | 7.1 | MSS |
| E00494 | Breast cancer | 30% | 1479 | 0.96 | 0.02 | MSS | 7.09 | MSS |
| E00557 | Breast cancer | 40% | 1525 | 0.94 | 0.02 | MSS | 5.14 | MSS |
| F02573 | Breast cancer | 45% | 674.4 | 0.92 | 0.01 | MSS | 6.73 | MSS |
| F02092 | Breast cancer | 40% | 753 | 0.94 | 0 | MSS | 6.2 | MSS |
| F00107 | Breast cancer | 20% | 1054 | 0.95 | 0.02 | MSS | 5.44 | MSS |
| F01141 | Breast cancer | 70% | 844.1 | 0.92 | 0.01 | MSS | 5.53 | MSS |
| F01409 | Breast cancer | 70% | 641.4 | 0.93 | 0 | MSS | 8.08 | MSS |
| F01898 | Breast cancer | 35% | 1264 | 0.96 | 0.01 | MSS | 4.07 | MSS |
| E00086 | Breast cancer | 55% | 828.7 | 0.93 | 0 | MSS | 7.81 | MSS |
| F02386 | Breast cancer | 55% | 1391 | 0.96 | 0.01 | MSS | 8.38 | MSS |
| D01394 | Breast cancer | 45% | 1003 | 0.94 | 0.01 | MSS | 5.18 | MSS |
| F02385 | Breast cancer | 50% | 1666 | 0.97 | 0.3 | MSS | 10.28 | MSS |
| D01491 | Breast cancer | 65% | 1206 | 0.95 | 0 | MSS | 5.63 | MSS |
| F00564 | Breast cancer | 80% | 1309 | 0.97 | 0 | MSS | 4.63 | MSS |
| F00201 | Breast cancer | 80% | 1518 | 0.96 | 0.02 | MSS | 3.56 | MSS |
| F01424 | Breast cancer | 10% | 1247 | 0.96 | 0 | MSS | 3.69 | MSS |
| F00486 | Breast cancer | 85% | 1605 | 0.98 | 0.04 | MSS | 3.62 | MSS |
| F01178 | Breast cancer | 25% | 1334 | 0.96 | 0.01 | MSS | 3.33 | MSS |
| F01459 | Breast cancer | 40% | 1265 | 0.95 | 0.02 | MSS | 4.31 | MSS |
| F01333 | Breast cancer | 60% | 1414 | 0.97 | 0.02 | MSS | 4.03 | MSS |
| F00110 | Breast cancer | 70% | 1812 | 0.97 | 0.02 | MSS | 6.42 | MSS |
| F00678 | Breast cancer | 50% | 1936 | 0.98 | 0 | MSS | 3.27 | MSS |
| F01362 | Breast cancer | 85% | 1634 | 0.94 | 0.03 | MSS | 5.79 | MSS |
| F01468 | Breast cancer | 60% | 1009 | 0.93 | 0.01 | MSS | 7.29 | MSS |
| F00817 | Breast cancer | NA | 2227 | 0.97 | 0.01 | MSS | 4.36 | MSS |
| F01130 | Breast cancer | 40% | 2128 | 0.98 | 0 | MSS | 3.09 | MSS |
| F01933 | Breast cancer | 15% | 1042 | 0.94 | 0.06 | MSS | 6.12 | MSS |
| F02365 | Breast cancer | 60% | 1498 | 0.98 | 0.01 | MSS | 5.63 | MSS |
| F02208 | Buccal cancer | 40% | 861.3 | 0.94 | 0.01 | MSS | 4.26 | MSS |
| D01571 | Bladder cancer | 65% | 886.3 | 0.95 | 0.02 | MSS | 5.46 | MSS |
| E00495 | Colon cancer | 55% | 1574 | 0.88 | 0.01 | MSS | 10.3 | MSS |
| F00369 | Oesophageal cancer | 50% | 2115 | 0.96 | 0.01 | MSS | 2.8 | MSS |
| F00716 | Prostate cancer | 75% | 2231 | 0.97 | 0.04 | MSS | 5.81 | MSI-L |
| F01155 | Rectum cancer | 60% | 708.6 | 0.92 | 0.01 | MSS | 4.17 | MSS |
| E00705 | Gastric Cancer | 40% | 1045 | 0.94 | 0.04 | MSS | 6.94 | MSS |
| F00426 | Uterine sarcoma | 90% | 1122 | 0.94 | 0.01 | MSS | 4.91 | MSS |
| D01878 | Cervical cancer | 60% | 1302 | 0.95 | 0.01 | MSS | 6.62 | MSS |
| D01878 | Cervical cancer | 60% | 1671 | 0.95 | 0.03 | MSS | 6.17 | MSS |
| D01870 | Cervical cancer | 40% | 876.5 | 0.94 | 0.01 | MSS | 10.31 | MSS |
| D01870 | Cervical cancer | 40% | 969.7 | 0.95 | 0 | MSS | 5.76 | MSS |
| E00208 | Cervical cancer | 55% | 840.8 | 0.94 | 0.01 | MSS | 11.47 | MSS |
| F01426 | Cervical cancer | 70% | 991.8 | 0.94 | 0 | MSS | 4.73 | MSS |
| F01287 | Cervical cancer | 25% | 1663 | 0.96 | 0.02 | MSS | 3.33 | MSS |
| E01827 | Cholangiocarcinoma | 25% | 1217 | 0.96 | 0.11 | MSS | 6.57 | MSS |
| F00381 | Cholangiocarcinoma | 60% | 1498 | 0.96 | 0.03 | MSS | 6.25 | MSS |
| E00224 | Cholangiocarcinoma | 60% | 883.4 | 0.94 | 0 | MSS | 5.12 | MSS |
| F00137 | Cholangiocarcinoma | 50% | 1021 | 0.96 | 0.01 | MSS | 3.89 | MSS |
| F01536 | Cholangiocarcinoma | 60% | 1068 | 0.95 | 0 | MSS | 4.1 | MSS |
| F02049 | Cholangiocarcinoma | 15% | 1348 | 0.96 | 0.01 | MSS | 4.49 | MSS |
| F02132 | Cholangiocarcinoma | 10% | 1949 | 0.98 | 0.01 | MSS | 6.38 | MSS |
| F02086 | Chondrosarcoma | 60% | 764.2 | 0.94 | 0.01 | MSS | 6.45 | MSS |
| E00167 | Brain cancer | 85% | 541.1 | 0.88 | 0 | MSS | 7.25 | MSI-L |
| F00844 | Ovarian cancer | 90% | 1100 | 0.97 | 0 | MSS | 3.34 | MSS |
| F02495 | Colon cancer | 30% | 1360 | 0.97 | 0.01 | MSS | 4.38 | MSS |
| F02346 | Colon cancer | 15% | 2403 | 0.98 | 0 | MSS | 9.65 | MSS |
| D01774 | Colon cancer | 60% | 706.8 | 0.94 | 0.03 | MSS | 5.48 | MSS |
| D01124 | Colon cancer | NA | 1488 | 0.95 | 0.02 | MSS | 4.11 | MSS |
| F00409 | Colon cancer | 15% | 1215 | 0.96 | 0.01 | MSS | 3.73 | MSS |
| F00556 | Colon cancer | 50% | 1227 | 0.95 | 0.01 | MSS | 3.36 | MSS |
| F00003 | Colon cancer | 35% | 1349 | 0.95 | 0.02 | MSS | 7.12 | MSS |
| F01115 | Colon cancer | 30% | 1727 | 0.96 | 0.04 | MSS | 4.39 | MSS |
| F02580 | Colon cancer | 15% | 1487 | 0.95 | 0.01 | MSS | 3.59 | MSS |
| F01402 | Colon cancer | 10% | 2262 | 0.98 | 0.03 | MSS | 4.14 | MSS |
| F02414 | Colon cancer | 35% | 1600 | 0.98 | 0.01 | MSS | 4.37 | MSS |
| F02071 | Colon cancer | 5% | 1430 | 0.95 | 0.02 | MSS | 6.45 | MSS |
| D00846 | NA | NA | 511.8 | 0.93 | 1 | MSI-H | 24.47 | MSI-H |
| D00923 | NA | NA | 608.8 | 0.94 | 1 | MSI-H | 17.92 | MSI-H |
| D00854 | NA | NA | 674.8 | 0.94 | 0.99 | MSI-H | 18.3 | MSI-H |
| D00927 | NA | NA | 712.1 | 0.94 | 1 | MSI-H | 19.81 | MSI-H |
| D00932 | NA | NA | 716.2 | 0.95 | 0.99 | MSI-H | 20.57 | MSI-H |
| D00938 | NA | NA | 755.2 | 0.95 | 1 | MSI-H | 25.18 | MSI-H |
| D00868 | NA | NA | 768.1 | 0.95 | 0.96 | MSI-H | 18.66 | MSI-H |
| D00881 | NA | NA | 788.4 | 0.95 | 1 | MSI-H | 17.57 | MSI-H |
| D00848 | NA | NA | 803.9 | 0.95 | 1 | MSI-H | 17.2 | MSI-H |
| D00900 | NA | NA | 815.9 | 0.95 | 0.02 | MSS | 6.21 | MSI-H |
| D00849 | NA | NA | 821.8 | 0.96 | 1 | MSI-H | 26.77 | MSI-H |
| D00895 | NA | NA | 828.2 | 0.95 | 0.97 | MSI-H | 17.29 | MSI-H |
| D00864 | NA | NA | 864.1 | 0.95 | 1 | MSI-H | 20.08 | MSI-H |
| D00918 | NA | NA | 906.7 | 0.96 | 1 | MSI-H | 13.6 | MSI-H |
| D00847 | NA | NA | 979.4 | 0.96 | 1 | MSI-H | 18.6 | MSI-H |
| D00893 | NA | NA | 986.2 | 0.96 | 0.99 | MSI-H | 18.48 | MSI-H |
| D00879 | NA | NA | 1054 | 0.96 | 0.99 | MSI-H | 12.45 | MSI-H |
| D00926 | NA | NA | 1116 | 0.97 | 0.99 | MSI-H | 20.11 | MSI-H |
| D00915 | NA | NA | 1330 | 0.95 | 0.79 | MSI-H | 20.98 | MSI-H |
| D00878 | NA | NA | 1377 | 0.96 | 0.87 | MSI-H | 14.44 | MSI-H |
| D00873 | NA | NA | 1498 | 0.96 | 0.16 | MSS | 10.17 | MSI-H |
| D00909 | NA | NA | 1575 | 0.96 | 0.05 | MSS | 13.73 | MSI-H |
| D00853 | NA | NA | 1995 | 0.97 | 0.76 | MSI-H | 9.26 | MSI-L |
| F00124 | Colorectal cancer | 90% | 1058 | 0.94 | 0.01 | MSS | 4.58 | MSI-L |
| F01012 | Colorectal cancer | 10% | 592.7 | 0.94 | 0.01 | MSS | 6.49 | MSS |
| F01495 | Colorectal cancer | 40% | 857.8 | 0.96 | 0 | MSS | 7.28 | MSS |
| F01460 | Colorectal cancer | 35% | 1731 | 0.97 | 0.01 | MSS | 5.44 | MSS |
| F01944 | Colorectal cancer | 15% | 3667 | 0.98 | 0.01 | MSS | 3.99 | MSI-L |
| F01080 | Rectal cancer | 60% | 1735 | 0.98 | 0 | MSS | 3.27 | MSS |
| F02388 | Cystic duct carcinoma | 40% | 1328 | 0.98 | 0.01 | MSS | 7.35 | MSS |
| F01194 | Dedifferentiated liposarcoma | 85% | 1144 | 0.94 | 0 | MSS | 4.17 | MSS |
| F00950 | Desmoid | 50% | 1675 | 0.97 | 0.01 | MSS | 2.92 | MSS |
| F00211 | Diffuse midline glioma | 70% | 945.6 | 0.95 | 0.07 | MSS | 4.31 | MSS |
| F00713 | Endometrial carcinoma | 50% | 1006 | 0.95 | 0.01 | MSS | 4.49 | MSS |
| F00318 | Endometrial cancer | 60% | 2074 | 0.97 | 0.06 | MSS | 1.83 | MSS |
| F01480 | Endometrial cancer | 30% | 948.9 | 0.94 | 0.23 | MSS | 11.22 | MSI-L |
| F01425 | Esophageal cancer | 20% | 965.4 | 0.93 | 0.02 | MSS | 4.1 | MSS |
| F01313 | Esophageal cancer | 25% | 629 | 0.94 | 0.03 | MSS | 11.74 | MSS |
| F00145 | Esophagus cancer | 10% | 1452 | 0.94 | 0.02 | MSS | 4.19 | MSS |
| F01089 | Esophageal cancer | 75% | 1146 | 0.93 | 0.01 | MSS | 5.74 | MSS |
| F01383 | Extraskeletal chondroblastic | 65% | 1708 | 0.95 | 0 | MSS | 3.74 | MSS |
| | osteosarcoma | | | | | | | |
| F01410 | Eyelid sebaceous carcinoma | 40% | 1019 | 0.96 | 0.09 | MSS | 3.53 | MSS |
| E02217 | Fallopian tube cancer | 85% | 1394 | 0.95 | 0.43 | MSS | 6.18 | MSI-H |
| F01537 | Gallbladder cancer | 40% | 1317 | 0.95 | 0.09 | MSS | 3.74 | MSS |
| D00304 | Gastric cancer | 13% | 836.6 | 0.95 | 0.03 | MSS | 9.21 | MSS |
| F02397 | Gastric cancer | 15% | 1326 | 0.98 | 0.01 | MSS | 7.4 | MSS |
| F00108 | Gastric cancer | 15% | 1571 | 0.97 | 0.02 | MSS | 7.26 | MSS |
| F00292 | Gastric cancer | 20% | 1809 | 0.98 | 0.04 | MSS | 5.47 | MSS |
| F01291 | Gastric cancer | 55% | 1156 | 0.97 | 0.05 | MSS | 4.77 | MSS |
| E00545 | Glioblastoma multiforme | 70% | 2408 | 0.96 | 0 | MSS | 4.22 | MSS |
| F01907 | Glioblastoma multiforme | 40% | 1389 | 0.97 | 0 | MSS | 5.08 | MSS |
| F01781 | Glioblastoma multiforme | 45% | 1370 | 0.95 | 0.01 | MSS | 5.66 | MSI-L |
| F00041 | Glioblastoma Multiforme | 65% | 1169 | 0.95 | 0.08 | MSS | 3.62 | MSS |
| F00766 | Glioblastoma Multiforme | 80% | 648.3 | 0.93 | 0.02 | MSS | 5.38 | MSS |
| F01073 | Glioblastoma multiforme | 50% | 1138 | 0.95 | 0.02 | MSS | 2.62 | MSS |
| F00345 | Glioblastoma multiforme | 60% | 1715 | 0.96 | 0 | MSS | 4.1 | MSS |
| F00120 | Glioblastoma multiforme | 45% | 1318 | 0.96 | 0.01 | MSS | 4.81 | MSI-L |
| F02320 | Gastrointestinal stromal tumor | 70% | 1114 | 0.95 | 0 | MSS | 5.61 | MSS |
| F00620 | Gastrointestinal stromal | 65% | 602.6 | 0.88 | 0.01 | MSS | 7.75 | MSS |
| | tumors (GIST) | | | | | | | |
| F02142 | Gastrointestinal stromal | 80% | 1187 | 0.96 | 0.01 | MSS | 5.24 | MSS |
| | tumor | | | | | | | |
| E00413 | Hepatocellular carcinoma | 70% | 1461 | 0.96 | 0.01 | MSS | 2.59 | MSS |
| F00052 | Hepatocellular carcinoma | 90% | 1240 | 0.96 | 0.03 | MSS | 3.68 | MSS |
| F01560 | Hepatocellular carcinoma | 60% | 1723 | 0.97 | 0.02 | MSS | 2.93 | MSS |
| F00881 | Hepatocellular carcinoma | 35% | 789.9 | 0.93 | 0.02 | MSS | 5.02 | MSS |
| F00882 | Cholangiocarcinoma | 40% | 835.6 | 0.94 | 0.03 | MSS | 5.7 | MSS |
| E00787 | High grade glioma | 40% | 729.1 | 0.93 | 0.01 | MSS | 3.85 | MSS |
| E00421 | Intima sarcoma | 90% | 1097 | 0.95 | 0.01 | MSS | 3.2 | MSS |
| E00421 | Intima sarcoma | 90% | 840.8 | 0.94 | 0.01 | MSS | 5.33 | MSS |
| F02066 | Invasive ductal carcinoma | 50% | 1065 | 0.96 | 0.02 | MSS | 5.6 | MSS |
| F01380 | Kidney cancer | 85% | 1627 | 0.97 | 0.03 | MSS | 4.92 | MSS |
| E01811 | Leiomyosarcoma | 45% | 1627 | 0.97 | 0.01 | MSS | 12.84 | MSS |
| F02519 | Leiomyosarcoma | 90% | 1298 | 0.96 | 0 | MSS | 9.94 | MSS |
| E00237 | Leiomyosarcoma | 85% | 1108 | 0.94 | 0.01 | MSS | 10.19 | MSS |
| F02519 | Leiomyosarcoma | 90% | 1298 | 0.96 | 0 | MSS | 9.94 | MSS |
| F02065 | Leiomyosarcoma | 75% | 1016 | 0.97 | 0.03 | MSS | 5.51 | MSS |
| F00988 | Leiomyosarcoma | 90% | 544.3 | 0.93 | 0.07 | MSS | 9.47 | MSS |
| D00546 | Liposarcoma | 98% | 1090 | 0.96 | 0.01 | MSS | 11.5 | MSS |
| F02026 | Liposarcoma | 90% | 1234 | 0.97 | 0 | MSS | 6.04 | MSS |
| F00942 | Liposarcoma | 75% | 1152 | 0.96 | 0.05 | MSS | 4.82 | MSS |
| F00805 | Liposarcoma | 40% | 1260 | 0.96 | 0.03 | MSS | 6.36 | MSS |
| F00962 | Liposarcoma | 90% | 1511 | 0.96 | 0 | MSS | 3.56 | MSS |
| F01154 | Liver cancer | NA | 1929 | 0.96 | 0.01 | MSS | 3.53 | MSS |
| F02019 | Liver angiosarcoma | 5% | 964.5 | 0.95 | 0.02 | MSS | 4.17 | MSS |
| F01489 | Liver cancer | 55% | 1219 | 0.97 | 0.01 | MSS | 3.49 | MSS |
| E00811 | Lung cancer | 10% | 660.2 | 0.95 | 0 | MSS | 5.93 | MSS |
| E00695 | Lung cancer | 5% | 861.3 | 0.94 | 0.01 | MSS | 5.47 | MSS |
| F00593 | Lung cancer | 40% | 948.3 | 0.95 | 0 | MSS | 9.51 | MSS |
| F00679 | Lung cancer | 0% | 1137 | 0.95 | 0.05 | MSS | 7.87 | MSS |
| E00704 | Lung Cancer | 60% | 1415 | 0.96 | 0.01 | MSS | 7.02 | MSS |
| F01960 | Lung cancer | 3% | 1474 | 0.96 | 0.22 | MSS | 8.67 | MSI-H |
| E00561 | Lung cancer | 85% | 1522 | 0.96 | 0.01 | MSS | 4.25 | MSS |
| E01825 | Lung cancer | 35% | 1598 | 0.97 | 0 | MSS | 6.49 | MSS |
| F01282 | Lung cancer | 50% | 1840 | 0.96 | 0.01 | MSS | 3.11 | MSS |
| F02483 | Lung cancer | 10% | 1297 | 0.96 | 0.01 | MSS | 9.29 | MSS |
| F00269 | Lung cancer | 2% | 811.8 | 0.95 | 0.03 | MSS | 7.33 | MSI-L |
| F00815 | Lung cancer | 60% | 1410 | 0.96 | 0.01 | MSS | 4.28 | MSS |
| F02497 | Lung cancer | 10% | 1491 | 0.96 | 0.01 | MSS | 3.56 | MSS |
| F00758 | Lung cancer | 60% | 1154 | 0.95 | 0.2 | MSS | 17.29 | MSS |
| F01494 | Lung cancer | 15% | 1329 | 0.96 | 0.01 | MSS | 6.2 | MSI-L |
| F02514 | Lung cancer | 40% | 2222 | 0.97 | 0.02 | MSS | 3.49 | MSS |
| F01321 | Lung cancer | 80% | 1498 | 0.97 | 0.04 | MSS | 5.45 | MSS |
| F01196 | Lung cancer | 35% | 1639 | 0.96 | 0.04 | MSS | 8.52 | MSS |
| F01151 | Lung cancer | 15% | 1813 | 0.96 | 0.03 | MSS | 2.79 | MSI-L |
| F02043 | Lung cancer | 30% | 1162 | 0.97 | 0.07 | MSS | 7.08 | MSS |
| F02483 | Lung cancer | 10% | 1297 | 0.96 | 0.01 | MSS | 9.29 | MSS |
| F02096 | Lung cancer | 55% | 1710 | 0.95 | 0.02 | MSS | 6.24 | MSS |
| D01492 | Lung cancer | 65% | 714.5 | 0.93 | 0.02 | MSS | 5.56 | MSS |
| F01782 | Lung cancer | 20% | 2187 | 0.96 | 0 | MSS | 6.15 | MSS |
| E00639 | Lung cancer | 45% | 1619 | 0.96 | 0.01 | MSS | 4.34 | MSS |
| F00946 | Lung cancer | 35% | 757.1 | 0.93 | 0.06 | MSS | 8.66 | MSS |
| F00251 | Lung cancer | 60% | 871.1 | 0.97 | 0.11 | MSS | 5.19 | MSS |
| F00762 | Lung cancer | 30% | 543.8 | 0.93 | 0.02 | MSS | 5.96 | MSS |
| F00159 | Lung cancer | 70% | 1085 | 0.95 | 0.02 | MSS | 3.93 | MSS |
| F00317 | Lung cancer | 50% | 1142 | 0.96 | 0.01 | MSS | 4.07 | MSS |
| F00790 | Lung cancer | 10% | 742.8 | 0.95 | 0.04 | MSS | 6.65 | MSS |
| F00141 | Lung cancer | 45% | 1302 | 0.96 | 0 | MSS | 4.26 | MSI-L |
| F00892 | Lung cancer | 40% | 1213 | 0.95 | 0.06 | MSS | 4.51 | MSS |
| F00895 | Lung cancer | 30% | 1256 | 0.96 | 0.08 | MSS | 4.98 | MSS |
| F00286 | Lung cancer | 15% | 1416 | 0.95 | 0.13 | MSS | 4.84 | MSS |
| F00654 | Lung cancer | 35% | 1471 | 0.95 | 0.01 | MSS | 3.37 | MSS |
| F00114 | Lung cancer | 25% | 1499 | 0.97 | 0.01 | MSS | 5.74 | MSS |
| F00479 | Lung cancer | 55% | 1511 | 0.95 | 0 | MSS | 5.45 | MSS |
| F01596 | Lung cancer | 60% | 921.1 | 0.94 | 0.01 | MSS | 4.34 | MSI-L |
| F00408 | Lung cancer | 60% | 1636 | 0.96 | 0.01 | MSS | 4.41 | MSS |
| F00994 | Lung cancer | 30% | 911.5 | 0.94 | 0.01 | MSS | 4.18 | MSS |
| F00038 | Lung cancer | 20% | 1930 | 0.98 | 0.01 | MSS | 3.24 | MSS |
| F00675 | Lung cancer | 15% | 1836 | 0.97 | 0.01 | MSS | 3.48 | MSS |
| F00610 | Lung cancer | 50% | 1613 | 0.98 | 0.01 | MSS | 3.26 | MSS |
| F00509 | Lung cancer | 40% | 1872 | 0.96 | 0 | MSS | 4.24 | MSS |
| F00559 | Lung cancer | 20% | 1947 | 0.98 | 0.12 | MSS | 3.43 | MSS |
| F02212 | Lung cancer | 25% | 697.5 | 0.94 | 0.03 | MSS | 9.35 | MSS |
| F00856 | Lung cancer | 85% | 1557 | 0.96 | 0.03 | MSS | 5.36 | MSS |
| F00413 | Lung cancer | 35% | 1998 | 0.98 | 0.03 | MSS | 4.55 | MSS |
| F01404 | Lung cancer | 25% | 927.3 | 0.96 | 0 | MSS | 6.65 | MSS |
| F02060 | Lung cancer | 20% | 857 | 0.96 | 0 | MSS | 6.48 | MSS |
| F01116 | Lung cancer | 10% | 1303 | 0.95 | 0 | MSS | 3.36 | MSS |
| F01290 | Lung cancer | 8% | 1284 | 0.96 | 0.01 | MSS | 5.52 | MSS |
| F00412 | Lung cancer | 25% | 2380 | 0.98 | 0.05 | MSS | 4.71 | MSS |
| F00894 | Lung cancer | 5% | 1863 | 0.96 | 0.08 | MSS | 2.99 | MSS |
| F00725 | Lung cancer | 40% | 2578 | 0.99 | 0.03 | MSS | 4.68 | MSS |
| F02579 | Lung cancer | 30% | 1345 | 0.96 | 0.01 | MSS | 3.02 | MSS |
| F02296 | Lung cancer | 10% | 1670 | 0.96 | 0 | MSS | 5.91 | MSS |
| F01125 | Lung cancer | 65% | 2208 | 0.97 | 0.02 | MSS | 4.03 | MSS |
| F01109 | Lung cancer | 80% | 1961 | 0.96 | 0.01 | MSS | 2.77 | MSS |
| F01163 | Pancreatic cancer | 10% | 1497 | 0.96 | 0.01 | MSS | 6.33 | MSS |
| E00784 | Sarcomatoid Carcinoma | 10% | 1339 | 0.95 | 0.02 | MSS | 4.1 | MSS |
| F00712 | Melanoma | 80% | 1611 | 0.97 | 0.01 | MSS | 14.18 | MSS |
| F00712 | Melanoma | 80% | 720.3 | 0.94 | 0.01 | MSS | 3.01 | MSS |
| F00040 | Meningioma | 85% | 2058 | 0.98 | 0.01 | MSS | 2.89 | MSS |
| F02202 | Ovarian cancer | NA | 1683 | 0.97 | 0.08 | MSS | 4.04 | MSS |
| E00674 | Breast Cancer | 40% | 3108 | 0.95 | 0.06 | MSS | 4.11 | MSS |
| E00674 | Breast Cancer | 40% | 1168 | 0.95 | 0 | MSS | 3.72 | MSS |
| F02451 | Epithelioid rhabdomyosarcoma | 75% | 1211 | 0.97 | 0.02 | MSS | 4.66 | MSS |
| F02478 | Melanoma | 25% | 1808 | 0.96 | 0.02 | MSS | 3.9 | MSS |
| F01075 | Pancreatic cancer | 20% | 2340 | 0.98 | 0.03 | MSS | 2.52 | MSS |
| F00793 | Tonsil cancer | 35% | 670.8 | 0.92 | 0.02 | MSS | 5.71 | MSS |
| F01305 | Metastasis of unknown | 35% | 1654 | 0.98 | 0.01 | MSS | 2.53 | MSS |
| | origin (MUO) | | | | | | | |
| F01576 | Metastasis of unknown | 10% | 1042 | 0.95 | 0.02 | MSS | 3.38 | MSS |
| | origin (MUO) | | | | | | | |
| F00585 | Nasopharyngeal cancer | 50% | 1482 | 0.96 | 0.02 | MSS | 7.42 | MSS |
| F01438 | Nasopharyngeal carcinoma | 30% | 1519 | 0.97 | 0.01 | MSS | 5.63 | MSS |
| F02024 | Lung cancer | 3% | 1718 | 0.97 | 0 | MSS | 9.44 | MSS |
| F02429 | Adenocarcinoma | 40% | 672.9 | 0.95 | 0.05 | MSS | 6.03 | MSS |
| F02329 | Lung cancer | 35% | 1508 | 0.94 | 0 | MSS | 7.9 | MSS |
| F00414 | NSCLC adenocarcinoma | 85% | 1062 | 0.97 | 0 | MSS | 4.39 | MSS |
| F00673 | NSCLC, adenocarcinoma | 65% | 995 | 0.93 | 0.04 | MSS | 6.8 | MSS |
| E00744 | Oesophageal Cancer | 25% | 1974 | 0.96 | 0 | MSS | 9.26 | MSS |
| F00288 | Oropharyngeal cancer | 50% | 838.3 | 0.95 | 0.03 | MSS | 4.29 | MSS |
| F01785 | Osteosarcoma | 35% | 1004 | 0.91 | 0 | MSS | 3.68 | MSS |
| F02155 | Ovarian cancer | 40% | 2518 | 0.99 | 0.03 | MSS | 3.93 | MSS |
| D01410 | Ovarian cancer | 70% | 757.5 | 0.94 | 0.38 | MSS | 15.75 | MSI-H |
| F01265 | Ovarian cancer | 60% | 1101 | 0.96 | 0.02 | MSS | 5.02 | MSS |
| E00608 | Endometrial cancer | 40% | 1611 | 0.96 | 0.04 | MSS | 2.41 | MSS |
| F02083 | Ovarian cancer | 50% | 837.3 | 0.94 | 0.01 | MSS | 5.64 | MSS |
| F00893 | Ovarian cancer | 35% | 759.7 | 0.94 | 0.01 | MSS | 5.63 | MSS |
| F02494 | Ovarian cancer | 85% | 1540 | 0.97 | 0.02 | MSS | 5.12 | MSS |
| F01200 | Ovarian cancer | 50% | 1174 | 0.94 | 0.01 | MSS | 4.73 | MSS |
| F01145 | Ovarian cancer | 95% | 2072 | 0.96 | 0.01 | MSS | 2.43 | MSS |
| F02390 | Ovarian cancer | 35% | 1081 | 0.94 | 0.11 | MSS | 9.04 | MSS |
| D00944 | Clear cell carcinoma | 85% | 1506 | 0.96 | 0.01 | MSS | 5.59 | MSI-L |
| F00298 | Ovarian cancer | 60% | 1001 | 0.96 | 0.05 | MSS | 3.7 | MSS |
| F00698 | Ovarian cancer | 60% | 834.9 | 0.95 | 0.03 | MSS | 7.52 | MSS |
| F00724 | Ovarian cancer | 20% | 1259 | 0.97 | 0.01 | MSS | 3.88 | MSS |
| F00920 | Ovarian cancer | 75% | 1483 | 0.97 | 0.04 | MSS | 6.42 | MSS |
| F00983 | Ovarian cancer | 60% | 764.5 | 0.96 | 0.01 | MSS | 8.6 | MSS |
| F01090 | Ovarian cancer | 90% | 1260 | 0.96 | 0.01 | MSS | 5.45 | MSS |
| F02070 | Ovarian cancer | 15% | 1281 | 0.96 | 0.01 | MSS | 4.08 | MSS |
| F01467 | Ovarian cancer | 35% | 1523 | 0.97 | 0.01 | MSS | 5.28 | MSI-L |
| F01763 | Ovarian cancer | NA | 1624 | 0.95 | 0.03 | MSS | 4.1 | MSS |
| F01400 | Ovarian cancer | 70% | 2197 | 0.98 | 0.01 | MSS | 5.1 | MSS |
| F02059 | Ovarian cancer | 75% | 1710 | 0.98 | 0.01 | MSS | 4.52 | MSS |
| F02010 | Ovarian cancer | 70% | 854.9 | 0.94 | 0 | MSS | 4.75 | MSS |
| F02194 | Ovarin cancer | 70% | 1051 | 0.95 | 0 | MSS | 5.28 | MSS |
| F00898 | Ovarian cancer | 80% | 841.6 | 0.92 | 0 | MSS | 5.8 | MSS |
| F00955 | Ovarian cancer | 45% | 1547 | 0.97 | 0.02 | MSS | 5.84 | MSS |
| F00900 | Ovarian cancer | 40% | 1771 | 0.96 | 0.05 | MSS | 5.22 | MSS |
| F02517 | Ovary cancer | 70% | 1774 | 0.98 | 0.04 | MSS | 4.39 | MSI-L |
| F02025 | Pancreatic cancer | 70% | 1646 | 0.97 | 0 | MSS | 7.13 | MSS |
| F00880 | Pancreatic cancer | 25% | 1165 | 0.95 | 0.04 | MSS | 5.59 | MSS |
| F00627 | Pancreatic cancer | 20% | 1624 | 0.96 | 0.01 | MSS | 3.58 | MSS |
| F01909 | Pancreatic cancer | 40% | 1231 | 0.96 | 0 | MSS | 5.33 | MSS |
| F00936 | Pancreatic cancer | 5% | 2249 | 0.98 | 0.02 | MSS | 5.23 | MSS |
| F01771 | Pancreatic cancer | 15% | 1912 | 0.97 | 0.01 | MSS | 4.6 | MSS |
| F02526 | Pancreatic cancer | 35% | 1359 | 0.97 | 0.01 | MSS | 8.82 | MSS |
| F02525 | Pancreatic cancer | 10% | 869.2 | 0.95 | 0 | MSS | 3.75 | MSS |
| E00666 | Pancreatic cancer | 5% | 1357 | 0.94 | 0.01 | MSS | 5.75 | MSS |
| F00081 | Pancreatic cancer | 80% | 909.1 | 0.95 | 0.01 | MSS | 9.63 | MSS |
| F01436 | Pancreatic cancer | 40% | 1782 | 0.97 | 0.09 | MSS | 5.28 | MSS |
| F01769 | Pancreatic cancer | 40% | 1557 | 0.96 | 0 | MSS | 4.53 | MSS |
| F00296 | Pancreatic cancer | 15% | 1299 | 0.97 | 0.03 | MSS | 6.04 | MSS |
| F00728 | Pancreatic cancer | 15% | 1570 | 0.97 | 0.01 | MSS | 14.15 | MSS |
| F00788 | Pancreatic cancer | 15% | 1490 | 0.97 | 0.02 | MSS | 3.62 | MSS |
| E01854 | Papillary Thyroid Carcinoma | 40% | 1538 | 0.97 | 0 | MSS | 5.96 | MSS |
| F00992 | Gastric cancer | 50% | 1156 | 0.96 | 0.01 | MSS | 3.31 | MSI-L |
| F00834 | Primary peritoneal serous | 40% | 695.5 | 0.95 | 0.01 | MSS | 4.15 | MSS |
| | carcinoma (PPSC) | | | | | | | |
| E01902 | prostate cancer | 5% | 1551 | 0.97 | 0.02 | MSS | 8.74 | MSS |
| F02364 | Prostate cancer | 25% | 1139 | 0.97 | 0.02 | MSS | 4.78 | MSS |
| F00044 | Prostate cancer | 35% | 2999 | 0.98 | 0.02 | MSS | 3.26 | MSS |
| E00755 | Renal cell carcinoma | 60% | 830.9 | 0.92 | 0 | MSS | 12.65 | MSS |
| E00755 | Renal cell carcinoma | 60% | 1279 | 0.94 | 0 | MSS | 3.48 | MSS |
| F00394 | Renal cell carcinoma | 85% | 1182 | 0.96 | 0.01 | MSS | 3.94 | MSS |
| F01081 | Rectal cancer | 10% | 1240 | 0.95 | 0 | MSS | 5.31 | MSS |
| F00326 | Rectal cancer | 50% | 1468 | 0.96 | 0.01 | MSS | 2.79 | MSS |
| F02135 | Rectal cancer | 10% | 2202 | 0.97 | 0.01 | MSS | 4.8 | MSS |
| F00586 | Rectum cancer | 25% | 1393 | 0.95 | 0 | MSS | 3.74 | MSS |
| F00119 | Renal cancer | 60% | 1837 | 0.96 | 0.01 | MSS | 4.45 | MSS |
| F00035 | Uterine cancer | 45% | 1554 | 0.98 | 0.06 | MSS | 3.45 | MSS |
| D02004 | Skin cancer | 65% | 805.9 | 0.93 | 0 | MSS | 13.93 | MSS |
| D02004 | Skin cancer | 65% | 526.5 | 0.91 | 0.01 | MSS | 5.27 | MSS |
| F02332 | Sarcoma | 5% | 2019 | 0.96 | 0.01 | MSS | 6.79 | MSS |
| F00987 | Sarcoma | 70% | 1701 | 0.97 | 0.01 | MSS | 3.28 | MSS |
| F00887 | Sarcoma | 40% | 555.2 | 0.93 | 0.03 | MSS | 6.65 | MSS |
| F00144 | Sarcoma | 60% | 1140 | 0.97 | 0.02 | MSS | 3.31 | MSS |
| F00603 | Sarcoma | 10% | 1608 | 0.97 | 0.1 | MSS | 4.25 | MSS |
| F01472 | Sarcoma | 50% | 1062 | 0.97 | 0.03 | MSS | 3.66 | MSS |
| F01520 | Sarcoma | 80% | 1080 | 0.95 | 0.01 | MSS | 3.95 | MSS |
| E01878 | Sigmoid cancer | 5% | 1435 | 0.92 | 0.01 | MSS | 6.12 | MSS |
| F02430 | Squamous cell carcinoma | 40% | 903.3 | 0.95 | 0 | MSS | 8.21 | MSS |
| E00318 | Stomach adenoacrinoma | 40% | 1456 | 0.96 | 0.02 | MSS | 4.81 | MSS |
| F01162 | Gastric cancer | 10% | 920.3 | 0.94 | 0.02 | MSS | 4.91 | MSS |
| F00171 | Gastric cancer | 10% | 1565 | 0.96 | 0.02 | MSS | 3.31 | MSS |
| F01377 | Gastric cancer | 75% | 1421 | 0.97 | 0.05 | MSS | 5.28 | MSS |
| F00274 | Submandibular gland cancer | 75% | 1012 | 0.97 | 0.01 | MSS | 5.17 | MSS |
| F00172 | Thymic cancer | 80% | 1273 | 0.95 | 0 | MSS | 3.56 | MSS |
| F01274 | Thymoma involvement | 35% | 1109 | 0.94 | 0.02 | MSS | 3.4 | MSS |
| F00245 | Thyriod cancer | 40% | 871.4 | 0.94 | 0.05 | MSS | 3.58 | MSS |
| F02375 | Breast cancer | 40% | 1242 | 0.94 | 0 | MSS | 4.96 | MSS |
| F00656 | Breast cancer | 85% | 2417 | 0.98 | 0.01 | MSS | 2.53 | MSS |
| F02369 | Tongue cancer | 40% | 1473 | 0.96 | 0.01 | MSS | 5.54 | MSS |
| E00764 | Tonsillar cancer | 50% | 1304 | 0.94 | 0.01 | MSS | 6.54 | MSS |
| E00764 | Tonsillar cancer | 50% | 1655 | 0.94 | 0 | MSS | 2.51 | MSS |
| F01546 | Transitional cell carcinoma | 45% | 680.3 | 0.95 | 0.02 | MSS | 6.38 | MSI-L |
| F01014 | Endometrioid adenocarcinoma | 40% | 1646 | 0.97 | 0.03 | MSS | 3.65 | MSS |
| F00624 | Uterus leiomyosarcoma | 40% | 1422 | 0.95 | 0.02 | MSS | 3.61 | MSS |
| F01281 | Hypopharyngeal Cancer | 60% | 2083 | 0.96 | 0 | MSS | 3.53 | MSS |
| F01414 | Oral Cancer | 35% | 521.5 | 0.92 | 0.03 | MSS | 11.35 | MSS |
| D01425 | Colon cancer | 60% | 858.9 | 0.95 | 0.01 | MSS | 5.83 | MSS |
| F01837 | Endometrial cancer | 25% | 1477 | 0.96 | 0.93 | MSI-H | 9.98 | MSI-H |
| F00956 | Endometrial cancer | 10% | 1485 | 0.95 | 0 | MSS | 2.64 | MSS |
| F02435 | Endometrial cancer | 60% | 1934 | 0.97 | 0.02 | MSS | 4.4 | MSS |
| F00891 | Endometrial cancer | 35% | 922.7 | 0.94 | 0.01 | MSS | 6.21 | MSS |
| F01833 | Leiomyosarcoma | 60% | 1693 | 0.97 | 0.03 | MSS | 4.04 | MSS |
| F00763 | Unknown primary | 10% | 1383 | 0.98 | 0.01 | MSS | 3.43 | MSS |
| F01174 | Unknown primary | 25% | 809 | 0.94 | 0.06 | MSS | 6.79 | MSS |
| F00811 | Unknown primary | 80% | 1318 | 0.97 | 0.03 | MSS | 6.07 | MSS |
| F00113 | Unknown primary | 60% | 1737 | 0.96 | 0.01 | MSS | 3.31 | MSS |
| F00765 | Breast cancer | 70% | 1272 | 0.97 | 0.01 | MSS | 4.62 | MSS |
| F01780 | Thyroid cancer | 10% | 703.7 | 0.92 | 0 | MSS | 5.98 | MSI-L |
| F02213 | Skin cancer | 60% | 907.3 | 0.97 | 0.01 | MSS | 4.66 | MSS |
| F02485 | Ovarian cancer | 40% | 1026 | 0.95 | 0.03 | MSS | 3.82 | MSS |
| F02415 | Ovarian cancer | 65% | 1581 | 0.96 | 0.09 | MSS | 15.76 | MSS |
| F01318 | Ovarian cancer | 20% | 1420 | 0.96 | 0 | MSS | 3.66 | MSS |
| F01267 | Ovarian cancer | 20% | 1729 | 0.96 | 0.03 | MSS | 3.53 | MSS |
| F00696 | Ovarian cancer | 70% | 828.9 | 0.94 | 0.01 | MSS | 5.36 | MSS |
| F02644 | Ovarian cancer | 50% | 2333 | 0.98 | 0.01 | MSS | 4.32 | MSS |
| F01519 | Ovarian cancer | 40% | 1407 | 0.97 | 0 | MSS | 4.61 | MSS |
| D00465 | Ovarian cancer | 80% | 1545 | 0.96 | 0.02 | MSS | 7.28 | MSS |
| F02189 | Ovarian cancer | 35% | 1528 | 0.98 | 0.06 | MSS | 3.82 | MSS |
| F02443 | Ovarian cancer/Endometrial | 70% | 1940 | 0.97 | 0 | MSS | 4.41 | MSS |
| | cancer | | | | | | | |
| F02100 | Cholangiocarcinoma | 45% | 1639 | 0.97 | 0.03 | MSS | 4.44 | MSS |
| E00771 | Breast Cancer | 50% | 963 | 0.94 | 0.02 | MSS | 14.75 | MSS |
| F00730 | Breast cancer | 35% | 1905 | 0.98 | 0.01 | MSS | 17.6 | MSS |
| F01173 | Breast cancer | 45% | 1282 | 0.95 | 0.05 | MSS | 4.36 | MSS |
| F00984 | Breast cancer | 35% | 1744 | 0.97 | 0.07 | MSS | 3.07 | MSS |
| E00771 | Breast Cancer | 50% | 1238 | 0.95 | 0.01 | MSS | 4.75 | MSS |
| F00985 | Breast cancer | 30% | 1463 | 0.96 | 0.09 | MSS | 3.94 | MSS |
| F01399 | Rectal cancer | 5% | 797.4 | 0.93 | 0 | MSS | 4.78 | MSS |
| F01401 | Rectal cancer | 30% | 1021 | 0.95 | 0 | MSS | 6.77 | MSI-L |
| F01118 | Lung cancer | NA | 1564 | 0.96 | 0.07 | MSS | 2.22 | MSS |
| F01539 | Lung cancer/Thyroid cancer | 20% | 1353 | 0.98 | 0.08 | MSS | 8.01 | MSS |
| F00421 | Gastric cancer | 50% | 1420 | 0.96 | 0.01 | MSS | 4.11 | MSS |
| F01598 | Gastric cancer | 15% | 965.3 | 0.96 | 0 | MSS | 6.02 | MSS |
| F01478 | Gastric cancer | 20% | 683.9 | 0.95 | 0.01 | MSS | 5.42 | MSS |
| F01482 | Gastric cancer | 15% | 760.4 | 0.94 | 0.01 | MSS | 5.83 | MSS |
| F02434 | Gastric cancer | 25% | 879.4 | 0.95 | 0.16 | MSS | 5.28 | MSS |
| F01929 | Esophageal cancer | 65% | 547.5 | 0.92 | 0 | MSS | 8.38 | MSS |
| F00396 | Unknown primary | 10% | 1741 | 0.97 | 0.01 | MSS | 3.81 | MSS |
| F02028 | Pancreatic cancer | 40% | 680.9 | 0.96 | 0.01 | MSS | 6.9 | MSS |
| F01198 | Pancreatic cancer | 40% | 1600 | 0.97 | 0.02 | MSS | 7.51 | MSS |
| F01903 | Pancreatic cancer | 15% | 1194 | 0.97 | 0 | MSS | 3.67 | MSS |
| F01912 | Pancreatic cancer | 10% | 1501 | 0.97 | 0 | MSS | 3.61 | MSS |
| F00360 | Pancreatic cancer | 20% | 1167 | 0.97 | 0.01 | MSS | 3.85 | MSS |
| F00789 | Pancreatic cancer | 35% | 861.8 | 0.94 | 0.03 | MSS | 4.95 | MSS |
| F00160 | Pancreatic cancer | 10% | 1472 | 0.95 | 0.04 | MSS | 2.82 | MSS |
| F01264 | Pancreatic cancer | 80% | 1383 | 0.98 | 0.03 | MSS | 5.8 | MSS |
| F01473 | Pancreatic cancer | 10% | 557.8 | 0.93 | 0.02 | MSS | 5.3 | MSS |
| F00674 | Pancreatic cancer | 65% | 2158 | 0.97 | 0.01 | MSS | 2.54 | MSS |
| F01582 | Pancreatic cancer | 30% | 771.1 | 0.93 | 0.01 | MSS | 5.27 | MSS |
| F01969 | Pancreatic cancer | 2% | 1669 | 0.98 | 0.01 | MSS | 4.01 | MSI-L |
| F01997 | Pancreatic cancer | 35% | 1013 | 0.94 | 0.01 | MSS | 7.13 | MSS |
| F01986 | Pancreatic cancer | 10% | 1923 | 0.99 | 0.03 | MSS | 4.89 | MSS |
| F01773 | Pancreatic cancer | 10% | 1450 | 0.97 | 0.04 | MSS | 4.55 | MSS |
| F01550 | Pancreatic cancer | 40% | 1781 | 0.96 | 0.01 | MSS | 5.57 | MSS |
| F02116 | Pancreatic cancer | 60% | 1966 | 0.98 | 0 | MSS | 3.09 | MSS |
| F02433 | Pancreatic cancer | 20% | 953.9 | 0.95 | 0.04 | MSS | 6.02 | MSS |
| F02527 | Pancreatic cancer | 10% | 2167 | 0.98 | 0.01 | MSS | 5.82 | MSS |
| F02041 | Pancreatic cancer | 40% | 1960 | 0.99 | 0.17 | MSS | 7.01 | MSS |
| F00868 | Thymic carcinoma | 25% | 911.8 | 0.95 | 0.01 | MSS | 4.92 | MSS |
| F02432 | Osteosarcoma | 90% | 1298 | 0.95 | 0 | MSS | 5.86 | MSS |
| F02646 | Osteosarcoma | 10% | 1453 | 0.93 | 0.01 | MSS | 4.84 | MSS |
| F00190 | Salivary gland cancer | 2% | 1620 | 0.96 | 0 | MSS | 3.9 | MSS |
| F01171 | Sarcoma | 35% | 1193 | 0.91 | 0 | MSS | 4.31 | MSS |
| F01427 | Kidney cancer | 80% | 1084 | 0.94 | 0 | MSS | 4.97 | MSS |
| E01792 | Melanoma | 40% | 1383 | 0.95 | 0.03 | MSS | 13.13 | MSS |
| E00467 | Peritoneal carcinoma | 40% | 996.4 | 0.94 | 0.01 | MSS | 5.44 | MSS |
| F01169 | Peritoneal cancer | 25% | 861.6 | 0.95 | 0.01 | MSS | 5.28 | MSS |
| F00129 | Peritoneal cancer | 60% | 1257 | 0.96 | 0.02 | MSS | 5.44 | MSS |
| F00803 | Bladder cancer | 80% | 704.9 | 0.94 | 0.03 | MSS | 3.2 | MSS |
| F02403 | Nasopharyngeal carcinoma | 85% | 1633 | 0.98 | 0.01 | MSS | 7.01 | MSS |
| F01176 | Sinus cancer | 40% | 1373 | 0.95 | 0.03 | MSS | 2.6 | MSS |
| F02171 | Head and Neck Cancers | 40% | 1302 | 0.93 | 0.01 | MSS | 4.54 | MSS |
| F00731 | Cholangiocarcinoma | 40% | 1525 | 0.97 | 0.99 | MSI-H | 15.72 | MSI-H |
| E00407 | Cholangiocarcinoma | NA | 1555 | 0.97 | 0 | MSS | 4.02 | MSS |
| F01172 | Cholangiocarcinoma | 25% | 944.7 | 0.93 | 0 | MSS | 3.03 | MSS |
| F00836 | Cholangiocarcinoma | 20% | 2087 | 0.97 | 0.01 | MSS | 3.68 | MSS |
| F01120 | Cholangiocarcinoma | 65% | 1250 | 0.97 | 0.02 | MSS | 2.93 | MSS |
| D00831 | Cholangiocarcinoma | 70% | 1498 | 0.97 | 0 | MSS | 3.85 | MSS |
| F00068 | Cholangiocarcinoma | 60% | 991.8 | 0.95 | 0.02 | MSS | 10.69 | MSS |
| F00493 | Cholangiocarcinoma | 2% | 1447 | 0.96 | 0.02 | MSS | 3.89 | MSS |
| F00727 | Cholangiocarcinoma | 20% | 1244 | 0.97 | 0.02 | MSS | 4.03 | MSS |
| F02115 | Cholangiocarcinoma | 10% | 3378 | 0.98 | 0.01 | MSS | 3.26 | MSS |
| F00246 | Cholangiocarcinoma | 40% | 1803 | 0.96 | 0.02 | MSS | 3.29 | MSS |
| F01288 | Cholangiocarcinoma | 65% | 1336 | 0.97 | 0.01 | MSS | 4.74 | MSS |
| F00976 | Cholangiocarcinoma | 20% | 1825 | 0.97 | 0.01 | MSS | 4.17 | MSS |
| F01060 | Cholangiocarcinoma | 10% | 1797 | 0.97 | 0 | MSS | 3.86 | MSS |
| F00186 | Gallbladder cancer | 40% | 1244 | 0.97 | 0.01 | MSS | 5.47 | MSS |
| F01266 | Lung cancer | 40% | 507.6 | 0.93 | 0.02 | MSS | 6.47 | MSS |
| F02384 | Prostate cancer | 35% | 1302 | 0.98 | 0.01 | MSS | 7.07 | MSS |
| ACT0744 | NA | NA | 554.2 | 0.92 | 1 | MSI-H | 27.02 | MSI-H |
| ACT0953 | NA | NA | 983.7 | 0.94 | 0.95 | MSI-H | 36.59 | MSI-H |
| ACT0893 | NA | NA | 1105 | 0.96 | 0 | MSS | 4.37 | MSS |
| ACT0897 | NA | NA | 1209 | 0.96 | 0.02 | MSS | 4.66 | MSS |
| ACT0894 | NA | NA | 1403 | 0.97 | 0.05 | MSS | 6.92 | MSS |
| ACT0887 | NA | NA | 1682 | 0.97 | 0.99 | MSI-H | 19.78 | MSI-H |
| ACT1217 | NA | NA | 1731 | 0.96 | 0.05 | MSS | 10.2 | MSS |
| F03491 | Anal cancer | 75% | 1394 | 0.96 | 0 | MSS | 4.98 | MSS |

TABLE 3
MSI Model Validation Results

| | | 5-marker MSI-PCR detection system | | |
|---|---|---|---|---|
| | | MSI-H | MSS | Total |
| MSI Model | MSI-H | 28 | 6 | 34 |
| | MSS | 2 | 403 | 405 |
| | Total | 30 | 409 | 439 |

TABLE 4
MSI Model Performance
Performance Summary

| Agreement Statistic | Point Estimate | Wilson Score 95% CI |
|---|---|---|
| PPA | 93% | 79%, 98% |
| NPA | 99% | 97%, 99% |
| PPV | 82% | 66%, 92% |
| NPV | 100% | 98%, 100% |

### EXAMPLE 3

### MSI detection for Samples of Different Tumor Purity

Total of three cancer cell lines with MSI-H were utilized (where they come from) for the determination of the lowest amount of tumor purity required to determine MSI status. These three cancer cell lines were diluted with their own matched normal cell to form a series of diluted samples with 100%, 80%, 50%, 40%, 30%, and 20% of tumor content. The MSI score for each of these samples is shown in Table 5.

TABLE 5
MSI status determined by MSI model for
cell lines of different tumor purity

| | Mean | Target base | Tumor/ | | |
|---|---|---|---|---|---|
| Cell | sequencing | coverage | Normal | MSI | MSI |
| line | depth | at 100x | percentage | score | status |
| RKO | 746.6 | 0.91 | 100%/0% | 0.85 | MSI-H |
| RKO | 623.3 | 0.92 | 80%/20% | 0.98 | MSI-H |
| RKO | 800.4 | 0.93 | 50%/50% | 1 | MSI-H |
| RKO | 824.1 | 0.92 | 40%/60% | 1 | MSI-H |
| RKO | 702.3 | 0.92 | 30%/70% | 1 | MSI-H |
| RKO | 712 | 0.92 | 20%/80% | 0.92 | MSI-H |
| C33A | 894.4 | 0.92 | 100%/0% | 0.99 | MSI-H |
| C33A | 687.3 | 0.92 | 80%/20% | 1 | MSI-H |
| C33A | 789.3 | 0.92 | 50%/50% | 1 | MSI-H |
| C33A | 763.8 | 0.92 | 40%/60% | 1 | MSI-H |
| C33A | 680.1 | 0.92 | 30%/70% | 0.99 | MSI-H |
| C33A | 694 | 0.92 | 20%/80% | 0.97 | MSI-H |
| SW48 | 1670 | 0.92 | 100%/0% | 1 | MSI-H |
| SW48 | 832.4 | 0.92 | 80%/20% | 1 | MSI-H |
| SW48 | 721.8 | 0.92 | 50%/50% | 1 | MSI-H |
| SW48 | 870.8 | 0.93 | 40%/60% | 1 | MSI-H |
| SW48 | 784.5 | 0.93 | 30%/70% | 0.99 | MSI-H |
| SW48 | 848 | 0.93 | 20%/80% | 0.66 | MSI-H |

1. A computer-implemented method of generating a model for predicting a microsatellite instability (MSI) status, comprising:
(a) collecting a clinical sample and an estimated MSI status data thereof;
(b) sequencing, through next-generation sequencing (NGS), at least six microsatellite loci of the clinical sample so as to generate a sequencing data;
(c) extracting a MSI feature from the sequencing data;
(d) training a machine learning model by mapping a MSI feature data with the estimated MSI status data; and
(e) outputting a trained machine learning model.

2. The computer-implemented method of claim 1, wherein the MSI feature data is calculated by a baseline.

3. The computer-implemented method of claim 2, wherein the baseline is established from a mean of each the MSI feature of each SSR region across normal samples.

4. The computer-implemented method of claim 2, wherein the baseline is established from a mean peak width of each SSR region across normal samples.

5. The computer-implemented method of claim 1, wherein the estimated MSI status data is retrieved from a cancer patient through an assay, comprising MSI-PCR assay, IHC or NGS-based MSI testing.

6. The computer-implemented method of claim 1, wherein the machine learning model comprises a logistic regression model, a random forest model, an extremely randomized trees model, a polynomial regression model, a linear regression model, a gradient descent model, or an extreme gradient boost model.

7. The computer-implemented method of claim 1, wherein the trained machine learning model comprises a defined weight of each microsatellite locus, and is predictive of the MSI status.

8. The computer-implemented method of claim 1, wherein the trained machine learning model comprises a defined weight of the MSI feature in each microsatellite locus and is predictive of the MSI status.

9. The computer-implemented method of claim 1, wherein the trained machine learning model has a cutoff value of 0.1, 0.15, 0.2, 0.25, 0.3, 0.35, 0.4, 0.45, or 0.5.

10. The computer-implemented method of claim 1, wherein the estimated MSI status data indicates microsatellite stability (MSS) or microsatellite instability-high (MSI-H).

11. A computer-implemented method for determining a MSI status, comprising:
(a) collecting a clinical sample from a subject;
(b) sequencing, through NGS, at least six microsatellite loci of the clinical sample so as to generate a sequencing data;
(c) extracting a MSI feature from the sequencing data;
(d) inputting a MSI feature data into the trained machine learning model of claim 1; and
(e) generating a computed MSI status.

12. The computer-implemented method of claim 11, further comprising step (f): outputting the computed MSI status data to an electronic storage medium or a display.

13. The computer-implemented method of claim 11, further comprising a step of identifying a treatment based on the computed MSI status data of the subject.

14. The computer-implemented method of claim 13, further comprising a step of administering a therapeutically effective amount of the treatment to the subject.

15. The computer-implemented method of claim 13, wherein the treatment comprises surgery, individual therapy, chemotherapy, radiation therapy, or immunotherapy.

16. The computer-implemented method of claim 15, wherein the immunotherapy comprises a step of administering a drug selected from the group consisting of pembrolizumab, nivolumab, MEDI0680, durvalumab and ipilimumab.

17. The computer-implemented method of claim 11, wherein the computed MSI status data indicates MSS or MSI-H.

18. The computer-implemented method of claim 1 or 11, wherein the microsatellite loci is at least 7, 10, 15, 20, 30, 40, 50, 100, 150, 200, 250, 300, 350, 400, 450, 500, 550 or 600 loci.

19. The computer-implemented method of claim 1 or 11, wherein the microsatellite loci with low coverage, unstable peak call, high variability in peak width or low weight are excluded.

20. The computer-implemented method of claim 19, wherein the microsatellite loci with low coverage has a read depth lower than 5x, 10x, 15x, 20x, 25x, 30x, 35x, 40x, 45x or 50x from a sample on a locus.

21. The computer-implemented method of claim 19, wherein the microsatellite loci with high variability in peak width has a peak width greater than 2 in 5 replicate runs, 3 in 6 replicate runs, 3 in 7 replicate runs, 3 in 8 replicate runs, 3 in 9 replicate runs, or 4 in 10 replicate runs.

22. The computer-implemented method of claim 1 or 11, wherein the MSI feature comprises peak width, peak height, peak location, simple sequence repeat (SSR) type or any combination thereof.

23. The computer-implemented method of claim 22, wherein the SSR type comprises mononucleotide with at least 10 repeats, dinucleotide with at least 6 repeats, trinucleotide with at least 5 repeats, tetranucleotide with at least 5 repeats, pentanucleotide with at least 5 repeats, and a complex nucleotide type of SEQ ID NOs: 1-37.

24. The computer-implemented method of claim 1 or 11, wherein the clinical sample originates from cell line, biopsy, primary tissue, frozen tissue, formalin-fixed paraffin-embedded (FFPE), liquid biopsy, blood, serum, plasma, buffy coat, body fluid, visceral fluid, ascites, paracentesis, cerebrospinal fluid, saliva, urine, tears, seminal fluid, vaginal fluid, aspirate, lavage, buccal swab, circulating tumor cell (CTC), cell-free DNA (cfDNA), circulating tumor DNA (ctDNA), DNA, RNA, nucleic acid, purified nucleic acid, purified DNA, or purified RNA.

25. The computer-implemented method of claim 1 or 11, wherein the clinical sample originates from a patient having cancer, solid tumor, hematologic malignancy, rare genetic disease, complex disease, diabetes, cardiovascular disease, liver disease, or neurological disease.

26. The computer-implemented method of claim 1 or 11, wherein a tumor purity of the clinical sample is at least 5, 10, 15, 20, 25, 30, 35, 40, 45, 50, 55, 60, 65, 70, 75, 80, 85, 90, 95, or 100%.

27. A system for determining a MSI status, comprising:
a data storage device storing instructions for determining characteristics of MSI status; and
a processor configured to execute instructions to perform a method including:
(a) training a machine learning model by mapping a training MSI feature data with a training estimated MSI status data;
(b) collecting a clinical sample from a subject;
(c) sequencing, through NGS, at least six microsatellite loci of the clinical sample so as to generate a sequencing data;
(d) computing, by using a trained machine learning model having a MSI feature data extracting from the sequencing data, an estimated MSI status data;
(e) generating a computed MSI status data; and
(f) outputting the computed MSI status data.

28. The system of claim 27, wherein the method further comprises step (g): identifying a treatment for the human subject based on the computed MSI status.

29. The system of claim 28, wherein the method further comprises step (h): administering a therapeutically effective amount of a treatment to the human subject.
